(19) United States
(12) Patent Application Publication
(10) Pub. No.: US 2009/0281030 A1
(43) Pub. Date: Nov. 12, 2009

(54) Genes Associated with Macular Degeneration

(75) Inventor: Josephine Hoh, New Haven, CT (US)

Correspondence Address: WOLF GREENFIELD & SACKS, P.C., 600 ATLANTIC AVENUE, BOSTON, MA, 02210-2206, US

(73) Assignee: Yale University, New Haven, CT (US)

(21) Appl. No.: 12/083,409

(22) PCT Filed: Oct. 11, 2006
(86) PCT No.: PCT/US2006/040071

§ 371 Date: Jun. 16, 2009

Related U.S. Application Data
(60) Provisional application No. 60/726,061, filed on Oct. 11, 2005.

(51) Int. Cl. A61K 38/16; C07H 21/00; G01N 33/48; G01N 33/68; B01L 3/00; A61K 31/7052; A61P 27/02

(52) U.S. Cl. 514/12; 536/24.31; 436/94; 436/86; 422/61; 514/44 R; 514/44 A

(57) ABSTRACT

Identification of variant genes correlated with age related macular degeneration, such as variant LOC387715, variant SYNPR and variant PDGFC; methods of identifying or aiding in identifying individuals at risk for developing age related macular degeneration.

### CROSS-REFERENCE TO RELATED APPLICATION

This application claims the benefit of U.S. Provisional Application No. 60/726,061 filed Oct. 11, 2005. The teachings of this referenced provisional application are incorporated by reference herein in their entirety.

This invention was made with United States government support under Grant Number HG000060 and Grant Number EY015771 from the National Institutes of Health. The United States government has certain rights in the invention.

### BACK GROUND OF THE INVENTION

Age-related macular degeneration (AMD) is the leading cause of blindness in the elderly in the developed world. Its incidence is increasing as lifespan lengthens and the elderly population expands (D. S. Friedman et al., Arch Opthalmol 122, 564 (2004)). It is a chronic disease characterized by progressive destruction of the retina's central region (macula), causing central field visual loss (J. Tuo, C. M. Bojanowski, C. C. Chan, Prog Retin Eye Res 23, 229 (2004)). One key characteristic of AMD is the formation of extracellular deposits called drusen that are concentrated in and around the macula behind the retina between the retina pigment epithelium (RPE) and choroid. The risk for developing AMD is determined by the complex interplay of genetic variants, many of which are as yet unidentified. Additional information about genetic determinants of AMD would be very valuable to the field.

### SUMMARY OF THE INVENTION

The present invention relates to identification of variations in human genes that are correlated with a predisposition to AMD. Such variations and the variant genes in which they occur are useful in identifying or aiding in identifying individuals at risk for developing AMD, as well as for diagnosing or aiding in the diagnosis of AMD. The invention also relates to methods for identifying or aiding in identifying individuals at risk for developing AMD, methods for diagnosing or aiding in the diagnosis of AMD, polynucleotides (e.g., probes, primers) useful in the methods, diagnostic kits containing probes or primers, methods of treating an individual at risk for or suffering from AMD and compositions useful for treating an individual at risk for or suffering from AMD.

Applicants analyzed genome-wide SNP genotyping data from individuals with AMD and individuals without AMD (controls) and looked for single associations at LOC387715 and other loci that appear to interact. One variant known to play a role in the risk of developing AMD is found in the gene LOC387715. As described herein, Applicants confirmed an association between LOC387115 and AMD. In addition, they provide evidence that the interaction of variants in the genes LOC387715, synaptoporin (SYNPR), and platelet-derived growth factor C(PDGFC), all of which are located in known AMD linkage peaks, contributes to AMD susceptibility. These interactions, along with the complement factor H (CFH) association Applicants previously identified, appear to account for considerable genetic risk for AMD.

In one embodiment, the present invention provides polynucleotides useful for the detection or aiding in the detection of a LOC387715 gene that is correlated with the occurrence of AMD in humans, a SYNPR gene that is correlated with the occurrence of AMD in humans and a PDGFC gene that is correlated with the occurrence of AMD -in humans. The phrases “correlated with the occurrence of AMD in humans” and “correlated with the occurrence of AMD” are used interchangeably herein. In specific embodiments, the invention relates to polynucleotides useful for detecting or aiding in detecting variations in each gene that are correlated with AMD in humans. In another embodiment, the present invention provides methods and compositions useful for identifying or aiding in identifying individuals at risk for developing AMD. In a further embodiment, the methods and compositions of the invention may be used for the treatment of an individual suffering from AMD or at risk for developing AMD. Also the subject of the invention are diagnostic kits for detecting a variant LOC387715 gene, a variant SYNPR gene and/or a variant PDGFC gene, alone or in combination, in a sample from an individual. Such kits can additionally be useful for detecting a variant CFI gene which comprises a variation in the CFH gene that is correlated with the occurrence of AMD. Such kits are useful in identifying or aiding in identifying individuals at risk for developing AMD, as well as for diagnosing or aiding in the diagnosis of AMD in an individual.

In specific embodiments, the invention provides isolated polynucleotides for the detection of a variant LOC387715 gene; isolated polynucleotides for the detection of a variant SYNPR gene; and isolated polynucleotides for the detection of a variant PDGFC gene. The isolated polynucleotide comprises a nucleic acid molecule that specifically detects a variation in the LOC387715 gene that is correlated with AMD in humans; a variation in the SYNPR gene that is correlated with AMD in humans; or variation in the PDGFC gene that is correlated with the occurrence of AMD in humans. Isolated polynucleotides are useful for detecting, in a sample from an individual, a variant LOC387715 gene, a variant SYNPR gene or a variant PDGFC gene that is correlated with AMD in humans.

The work described herein provides strong evidence of genetic interactions at three loci on distinct chromosomes for susceptibility to AMD. Variant(s) of LOC387715 function in conjunction with variant(s) of synaptoporin (SYNPR) and variant(s) of platelet derived growth factor C(PDGFC). In contrast, CFH variants independently contribute to an individual's genetic risk of developing AMD. The estimated PAR reached a level (0.55 to 0.71) that is as high as a previously estimated level (0.46 to 0.71) of the genetic contribution to AMD (16), supporting the hypothesis that reported genetic network(s) capture a substantial portion of the genetic risk for AMD, for example, in populations of European descent. The contribution of these genetic network(s) to AMD susceptibility in other populations can be confirmed or determined using the methods described herein.

### DETAILED DESCRIPTION OF THE INVENTION

### Overview

As described herein, Applicants have investigated the LOC387715 locus, independently and in concert with other genes, in genome-wide association data obtained from genotyping individuals from the age-related eye disease study (AREDS) for more than 100,000 single nucleotides polymorphisms (SNPs) (AREDS Research Group, Ophthamology 107, 2224 (2000)). As also described herein, results of that investigation have shown the association of variants of three genes (LOC387715, synaptoporin (SYNPR), and platelet derived growth factor C(PDGFC) with the development of AMD and support the role of their interaction in susceptibility to AMD. Variants of the three genes are represented herein, respectively as vLOC387715, vSYNPR, and vPDGFC. These interactions, along with the complement factor H(CFH) association Applicants previously identified, appear to account for considerable genetic risk for AMD. Assessment of variants of the three genes that are associated with the occurrence of AMD and assessment of variants of the three genes in combination with assessment of a variant CFH gene that is correlated with the occurrence of AMD are useful in identifying or aiding in identifying an individual at risk for developing AMD, as well as in diagnosing or aiding in diagnosing AMD in an individual (e.g., a human).

Variations in the LOC387715 gene, variations in the SYNPR gene and variations in the PDGFC gene shown to be correlated (associated) with AMD in humans are useful for the early diagnosis and treatment of individuals predisposed to AMD. The determination of the genetic constitution of the LOC387715 gene, the SYNPR gene, and the PDGFC gene in an individual (human) is useful in treating AMD at earlier stages, or even before an individual displays any symptoms of AMD. Furthermore, diagnostic tests to genotype LOC387715, SYNPR, and PDGFC may allow individuals, such as those shown to be at risk for developing AMD, to alter their behavior to reduce environmental risks that contribute to the development of AMD (e.g., smoking) and, as a result, reduce their risk of developing AMD, reduce the severity of AMD and/or delay its onset.

In one embodiment, the present invention relates to the identification of vLOC387715 gene(s), vSYNPR gene(s), and vPDGFC gene(s) that are correlated with the occurrence of AMD a predisposition to (increased likelihood of developing) AMD in humans. These variants are useful in identifying or aiding in identifying individuals at risk for developing AMD, as well as for diagnosing or aiding in the diagnosis of AMD. The invention also relates to methods for identifying or aiding in identifying individuals at risk for developing AMD, methods and compositions for detecting such variations that predispose a human to AMD, methods for diagnosing or aiding in the diagnosis of AMD, polynucleotides (e.g., probes, primers) useful in the methods, diagnostic kits that contain probes or primers and are useful in the methods of this invention, methods of treating an individual at risk for or suffering from AMD and compositions useful for treating an individual at risk for or suffering from AMD. Variants of the three genes shown herein to interact can be assessed in the methods of the present invention alone (without assessment of other factor(s), such as without assessment of a variant CFH gene that is correlated with the occurrence of AMD) or in combination with assessment of additional factor(s), such as in combination with assessment of a variant CFH gene that is correlated with AMD or assessment of clinical.

LOC387715, SYNPR, and PDGFC genes can be cDNA or the genomic form of the gene, which may include upstream and downstream regulatory sequences. See, for example, homosapiens gene LOC387715 entry at http://www.ncbi.nlm.nih.gov; synaptoporin (rat protein P22831 EMBL and SYNPR synaptorin Gene ID 66030 Entrez Gene at http://rat.embl.de; SYNPR_MOUSE Q8BGN8 at http://us.expasy.org; human protein Q8TBG9—Synaptoporin EMBL and SYNPR synaptoporin [Homosapiens] at http://harvester.embl.de); PDGFC (Genbank accession AF336376; Utela et al. Circulation 2001; 103:2242-2247) for examples of sequences, which are not intended to be limiting in any way. Polynucleotide probes and primers of the invention may hybridize to any contiguous portion of one of the three genes (LOC387715, SNYPR or PDGFC or to any contiguous portion of one of the three gene variants (vLOC387715, vSNYPR or vPDGFC). The LOC387715, SYNPR, AND PDGFC genes may further include sequences located adjacent to the coding region on both the 5′ and 3′ ends for a distance of about 1-2 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences.

Also the subject of the invention are isolated vLOC387715 polypeptides; isolated vSYNPR polypeptides; and isolated vPDGFC polypeptide and their use in methods of the present invention, such as methods of identifying or aiding in identifying individuals at risk for developing AMD, methods for detecting such variations that predispose a human to AMD, and methods for diagnosing or aiding in the diagnosis of AMD vLOC387715 polypeptidesequences include human polypeptidesequences, such as the Ala69Ser change encoded by the coding change in the LOC387715 gene described by Fisher and coworkers (8) and nonhuman (e.g., rat, mouse) polypeptidesequences. Similarly, vSYNPR polypeptides and vPDGFC polypeptides include human and nonhuman sequences. The LOC387715, SYNPR, and PDGFC polypeptides can be encoded by a full length coding sequence or by any portion of the coding sequence and vLOC387715, vSYNPR, and vPDGFC polypeptides can be encoded by a full length coding sequence or by any portion of the coding sequence, as long as the encoded polypeptide has the desired activity or functional property (e.g., enzymatic activity, ligand binding, signal transduction).

LOC387715, SYNPR, and PDGFC polynucleotide probes and primers

In certain embodiments, the invention provides isolated and/or recombinant polynucleotides that specifically detect a variation in the LOC387715 gene that is correlated with the occurrence of AMD, a variation in the SYNPR gene that is correlated with the occurrence of AMD, or a variation in the PDGFC gene that is correlated with the occurrence of AMD or a combination thereof. Polynucleotide probes of the invention hybridize to a variation (referred to as a variation of interest) in such a LOC387715 gene, SYNPR gene, or PDGFC gene, and the flanking sequence, in a specific manner and thus typically have a sequence which is fully or partially complementary to the sequence of the variation and the flanking region. Polynucleotide probes of the invention may hybridize to a segment of a gene or to DNA that comprises a variation of interest such that the variation aligns with a central portion of the probe or with another portion of the probe, such as a terminal portion of the probe. In one embodiment, an isolated polynucleotide probe of the invention hybridizes, under stringent conditions, to a nucleic acid molecule comprising a variant LOC387715 gene that is correlated with AMD, a variant SYNPR gene that is correlated with the occurrence of AMD, or a variant PDGFC gene that is correlated with the occurrence of AMD in humans, or a portion or allelic variant thereof. In another embodiment, an isolated polynucleotide probe of the invention hybridizes, under stringent conditions, to a nucleic acid molecule comprising at least 10 contiguous nucleotides of a LOC387715 gene, a SYNPR gene, a PDGFC gene, a variant LOC387715 gene that is correlated with AMD, a variant SYNPR gene that is correlated with AMD, a variant PDGFC gene that is correlated with AMD or an allelic variant thereof, wherein the nucleic acid molecule comprises a variation that is correlated with the occurrence of AMD in humans.

In certain embodiments, a polynucleotide probe of the invention is an allele-specific probe. The design and use of allele-specific probes for analyzing polymorphisms is described by e.g., Saiki et al., Nature 324:163-166 (1986); Dattagupta, EP 235726; and Saiki WO 89/11548. Allele-specific probes can be designed to hybridize to a segment of a target DNA from one individual but do not hybridize to the corresponding segment from another individual due to the presence of different polymorphic forms or variations in the respective segments from the two individuals. Hybridization conditions should be sufficiently stringent such that there is a significant difference in hybridization intensity between alleles. In some embodiments ,a probe hybridizes to only one of the alleles.

A variety of variations in the LOC387715 gene, the SYNPR gene, and the PDGFC gene or any combination of such variations that predispose an individual to AMD may be detected by the methods and polynucleotides described herein. For example, any nucleotide polymorphism of a coding region, exon, exon-intron boundary, signal peptide, 5-prime untranslated region, promoter region, enhancer sequence, 3-prime untranslated region or intron that is associated with AMD in humans can be detected. These polymorphisms include, but are not limited to, changes that: alter the amino acid sequence of the proteins encoded by the LOC387715 gene, the SYNPR gene, and/or the PDGFC gene, produce alternative splice products, create truncated products, introduce a premature stop codon, introduce a cryptic exon, alter the degree or expression to a greater or lesser extent, alter tissue specificity of expression of the gene, introduce changes in the tertiary structure of the proteins encoded by LOC387715, SYNPR, or PDGFC, introduce changes in the binding affinity or specificity of the proteins expressed by LOC387715, SYNPR, or PDGFC or alter the function of the proteins encoded by LOC387715, SYNPR, or PDGFC. In a specific embodiment, the variation in the LOC387715 gene encodes an amino acid other than alanine (e.g., serine) at position 69 of LOC387715 protein. Other variant genes, such as those in which the variation is in a coding region (e.g., variations that encode an amino acid other than amino acid present at the corresponding position in a LOC387715 gene that is not correlated with AMD, at the corresponding position in a SYNPR gene that is not correlated with AMD or in a PDGFC gene-that is not correlated with AMD)) can be detected using the methods and compositions described herein. Alternatively, variant genes in which the variation is in a noncoding region, may be detected using the methods and compositions described herein. The subject polynucleotides are further understood to include polynucleotides that are variants of the polynucleotides described herein, provided that the variant polynucleotides maintain their ability to specifically detect a variation in the LOC387715 gene, the SYNPR gene or the PDGFC gene that is correlated with the occurrence of AMD. Variant polynucleotides may include, for example, sequences that differ by one or more nucleotide substitutions, additions or deletions.

In certain embodiments, the isolated polynucleotide is a probe that hybridizes, under stringent conditions, to a variation in the LOC387715 gene that is correlated with the occurrence of AMD in humans, a variation in the SYNPR gene that is correlated with the occurrence of AMD in humans, or a variation in the PDGFC gene that is correlated with the occurrence of AMD in humans. The term “probe” refers to a polynucleotide that is capable of hybridizing to another nucleic acid of interest. The polynucleotide may be naturally occurring, as in a purified restriction digest, or it may be produced synthetically, recombinantly or by nucleic acid amplification (e.g., PCR amplification).

It is well known in the art how to perform hybridization experiments with nucleic acid molecules. The skilled artisan is familiar with hybridization conditions. Such hybridization conditions are referred to in standard text books, such as Molecular Cloning: A Laboratory Manual, Cold Spring Harbor Laboratory (2001); and Current Protocols in Molecular Biology, eds. Ausubel et al., John Wiley & Sons (1992). Particularly useful in methods of the present invention are polynucleotides which hybridize to a variation in the LOC387715 gene that is correlated with the occurrence of AMD in humans, a variation in the SYNPR gene that is correlated with the occurrence of AMD in humans, or a variation in the PDGFC gene that is correlated with the occurrence of AMD in humans or a region of a variant LOC387715, SYNPR, or PDGFC gene, under stringent conditions. Under stringent conditions, a polynucleotide that hybridizes to a variant LOC387715 gene that is correlated with the occurrence of AMD in humans, a variant SYNPR gene that is correlated with the occurrence of AMD in humans, or a variant PDGFC gene that is correlated with the occurrence of AMD in humans does not hybridize to the corresponding LOC387715, SYNPR, or PDGFC gene that does not include the variation of interest.

Nucleic acid hybridization is affected by such conditions as salt concentration, temperature, organic solvents, base composition, length of the complementary strands, and the number of nucleotide base mismatches between the hybridizing nucleic acids, as will readily be appreciated by those skilled in the art. Stringent temperature conditions will generally include temperatures in excess of 30° C., or may be in excess of 37° C. or 45° C. Stringency increases with temperature. For example, temperatures greater than 45° C. are highly stringent conditions. Stringent salt conditions will ordinarily be less than 1000 mM, or may be less than 500 mM or 200 mM. For example, one could perform the hybridization at 6.0× sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. Particularly useful in methods of the present invention are polynucleotides that are capable of hybridizing to a variant LOC387715 gene that is correlated with the occurrence of AMD in humans, a variant SYNPR gene that is correlated with the occurrence of AMD in humans, or a variant PDGFC gene that is correlated with the occurrence of AMD in humans, or a region of a variant LOC387715, SYNPR, or PDGFC gene, under stringent conditions. It is understood, however, that the appropriate stringency conditions may be varied to promote DNA hybridization. In certain embodiments, polynucleotides of the present invention hybridize to a variant LOC387715 gene that is correlated with the occurrence of AMD in humans, a variant SYNPR gene that is correlated with the occurrence of AMD in humans , or a variant PDGFC gene that is correlated with the occurrence of AND in human, or a region of such a variant LOC387715 gene, a variant SYNPR gene, or a variant PDGFC gene, under highly stringent conditions. Under stringent conditions, a polynucleotide that hybridizes to a variation in the LOC387715 gene, a variation in the SYNPR gene, or a variation in the PDGFC gene does not hybridize to the corresponding LOC387715, SYNPR, or PDGFC gene that does not include the variation of interest. In one embodiment, the invention provides nucleic acids that hybridize under low stringency conditions of 6.0×SSC at room temperature followed by a wash at 2.0×SSC at room temperature. The combination of parameters, however, is much more important than the measure of any single parameter. See, e.g., Wetmur and Davidson, 1968. Probe sequences-may also hybridize specifically to duplex DNA under certain conditions to form triplex or higher order DNA complexes. The preparation of such probes and suitable hybridization conditions are well-known in the art.

A polynucleotide probe or primer of the present invention may be labeled so that it is detectable in a variety of detection systems, including, but not limited, to enzyme (e.g., ELISA, as well as enzyme-based histochemical assays), fluorescent, radioactive, chemical, and luminescent systems. A polynucleotide probe or primer of the present invention may further include a quencher moiety that, when placed in proximity to a label (e.g., a fluorescent label), causes there to be little or no signal from the label. Detection of the label may be performed by direct or indirect means (e.g., via a biotin/avidin or a biotin/streptavidin linkage). It is not intended that the present invention be limited to any particular detection system or label.

In another embodiment, the isolated polynucleotide of the invention is a primer that hybridizes, under stringent conditions, adjacent, upstream, or downstream to a variation in a LOC387715 gene, a SYNPR gene, or a PDGFC gene that is correlated with the occurrence of AMD in humans. The isolated polynucleotide may hybridize, under stringent conditions, to a nucleic acid molecule comprising all or a portion of a variant LOC387715, variant SYNPR, or variant PDGFC gene that is correlated with the occurrence of AMD in humans. Alternatively, the isolated polynucleotide primer may hybridize, under stringent conditions, to a nucleic acid molecule comprising at least 50 contiguous nucleotides of a variant LOC387715, variant SYNPR, or variant PDGFC gene that is correlated with the occurrence of AMD in humans. For example, a polynucleotide primer of the invention can hybridize adjacent, upstream, or downstream to the region of the LOC387715 gene that encodes amino acid 69 of the encoded protein.

As used herein, the term “primer” refers to a polynucleotide that is capable of acting as a point of initiation of nucleic acid synthesis when placed under conditions in which synthesis of a primer extension product that is complementary to a nucleic acid strand occurs (for example, in the presence of nucleotides, an inducing agent such as DNA polymerase, and suitable temperature, pH, and electrolyte concentration). Alternatively, the primer may be capable of ligating to a proximal nucleic acid when placed under conditions in which ligation of two unlinked nucleic acids occurs (for example, in the presence of a proximal nucleic acid, an inducing agent such as DNA ligase, and suitable temperature, pH, and electrolyte concentration). A polynucleotide primer of the invention may be naturally occurring, as in a purified restriction digest, or may be produced synthetically. The primer is preferably single stranded for maximum efficiency in amplification, but may alternatively be double stranded. If double stranded, the primer is first treated to separate its strands before being used. Preferably, the primer is an oligodeoxyribonucleotide. The exact lengths of the primers will depend on many factors, including temperature, source of primer and the use of the method. In certain embodiments, the polynucleotide primer of the invention is at least 10 nucleotides long and hybridizes to one side or the other of a variation in the LOC387715, SYNPR, or PDGFC gene that is correlated with the occurrence of AMD in humans. The subject polynucleotides may contain alterations, such as one or more nucleotide substitutions, additions or deletions, provided they hybridize to their target variant LOC387715, SYNPR, and/or PDGFC gene with substantially the same degree of specificity.

In one embodiment, the invention provides a pair of primers that specifically detect a variation in the LOC387715 gene that is correlated with AMD, a variation in the SYNPR gene that is associated with AMD, or a variation in the PDGFC gene that is correlated with the occurrence of AMD. In such a case, the first primer hybridizes upstream from the variation and a second primer hybridizes downstream from the variation. For example, one of the primers hybridizes to one strand of a region of DNA that comprises a variation in the LOC387715 gene, a variation in the SYNPR gene that is correlated with AMD or a variation in the PDGFC gene that is correlated with the occurrence of AMD, and the second primer hybridizes to the complementary strand of a region of DNA that comprises a variation in the LOC387715 gene that is correlated with AMD, a variation in the SYNPR gene that-is correlated with AMD, or a variation in the PDGFC gene that is correlated with the occurrence of AMD. As used herein, the term “region of DNA” refers to a sub-chromosomal length of DNA.

In another embodiment, the invention provides an allele-specific primer that hybridizes to a site on target DNA that overlaps a variation in the LOC387715 gene that is correlated with AMD, a variation in the SYNPR gene that is correlated with AMD or a variation in the PDGFC gene that is correlated with the occurrence of AMD. An allele-specific primer of the invention only primes amplification of an allelic form to which the primer exhibits perfect complementarity. This primer may be used, for examples in conjunction with a second primer which hybridizes at a distal site. Amplification can thus proceed from the two primers, resulting in a detectable product that indicates the presence of a variant LOC387715 gene that is correlated with the occurrence of AMD, a variant SYNPR gene that is correlated with the occurrence of AMD, or a variant PDGFC gene that is correlated with the occurrence of AMD.

Detection Assays

In certain embodiments, the invention relates to polynucleotides useful for detecting a variation in a LOC387715, SYNPR, or PDGFC gene that is correlated with the occurrence of age related macular degeneration. Preferably, these polynucleotides are capable of hybridizing, under stringent hybridization conditions, to a region of DNA that comprises a variation in the LOC387715 gene, a variation in the SYNPR gene, or a variation in the PDGFC gene that is correlated with the occurrence of age related macular degeneration.

The polynucleotides of the invention may be used in any assay that permits detection of a variation in the LOC387715, SYNPR, or PDGFC gene that is correlated with the occurrence of AMD. Such methods may encompass, for example, DNA sequencing, hybridization, ligation, or primer extension methods. Furthermore, any combination of these methods may be utilized in the invention.

In one embodiment, the presence of a variation in the LOC387715, SYNPR, PDGFC gene or combination thereof that is correlated with the occurrence of AMD is detected and/or determined by DNA sequencing. DNA sequence determination may be performed by standard methods such as dideoxy chain termination technology and gel-electrophoresis, or by other methods such as by pyrosequencing (Biotage AB, Uppsala, Sweden). For example, DNA sequencing by dideoxy chain termination may be performed using unlabeled primers and labeled (e.g., fluorescent or radioactive) terminators. Alternatively, sequencing may be performed using labeled primers and unlabeled terminators. The nucleic acid sequence of the DNA in the sample can be compared to the nucleic acid sequence of wildtype DNA or DNA that does not comprise a variation correlated with the occurrence of AMD to determine whether a variation in the LOC387715 gene that is correlated with AMD, a variation in the SYNPR gene that is correlated with AMD, a variation in the PDGFC gene that is correlated with the occurrence of AMD or a combination of such variations is present.

In another embodiment, the presence of a variation in the LOC387715 gene that is correlated with the occurrence of AMD, a variation in the SYNPR gene that is correlated with the occurrence of AMD , a variation in the PDGFC gene that is correlated with the occurrence of AMD or a combination thereof is detected and/or determined by hybridization. In one embodiment, a polynucleotide probe hybridizes to a variation in the LOC387715 gene, SYNPR gene, or PDGFC gene that is correlated with AMD and flanking nucleotides, but not to a LOC387715, SYNPR, or PDGFC gene that does not contain a variation that is correlated with AMD. The polynucleotide probe may comprise nucleotides that are fluorescently, radioactively, or chemically labeled to facilitate detection of hybridization. Hybridization may be performed and detected by standard methods known in the art, such as by Northern blotting, Southern blotting, fluorescent in situ hybridization (FISH), or by hybridization to polynucleotides immobilized on a solid support, such as a DNA array or microarray. As used herein, the terms “DNA array,” and “microarray” refer to an ordered arrangement of hybridizable array elements. The array elements are arranged so that there are preferably at least one or more different array elements immobilized on a substrate surface. The hybridization signal from each of the array elements is individually distinguishable.

In another embodiment, the presence of a variation in the LOC387715 gene that is correlated with the occurrence of AMD is detected and/or determined by hybridization.

In another embodiment, the presence of a variation in the SYNPR gene that is correlated with the occurrence of AMD is detected and/or determined by hybridization.

In another embodiment, the presence of a variation in the PDGFC gene that is correlated with the occurrence of AMD is detected and/or determined by hybridization.

In a specific embodiment, the polynucleotide probe is used to hybridize genomic DNA by FISH. FISH can be used, for example, in metaphase cells, to detect a deletion in genomic DNA. Genomic DNA is denatured to separate the complimentary strands within the DNA double helix structure. The polynucleotide probe of the invention is then added to the denatured genomic DNA. If a variation in the LOC387715 gene that is correlated with the occurrence of AMD, a variation in the SYNPR gene that is correlated with the occurrence of AMD, or a variation in the PDGFC gene that is correlated with the occurrence of AMD is present, the probe will hybridize to the genomic DNA The probe signal (e.g., fluorescence) can then be detected through a fluorescent microscope for the presence of absence of signal. The absence of signal, therefore, indicates the absence of a variation in the respective gene that is correlated with the occurrence of AMD. In another specific embodiment, a labeled polynucleotide probe is applied to immobilized polynucleotides on a DNA array. Hybridization may be detected, for example, by measuring the intensity of the labeled probe remaining on the DNA array after washing. The polynucleotides of the invention may also be used in commercial assays, such as the Taqman assay (Applied Biosystems, Foster City, Calif.).

In another embodiment, the presence of a variation in the LOC387715 gene that is correlated with the occurrence of AMD, a variation in the SYNPR that is correlated with the occurrence of AMD, or a variation in the PDGFC gene that is correlated with the occurrence of AMD is detected and/or determined by primer extension with DNA polymerase. In one embodiment, a polynucleotide primer of the invention hybridizes immediately adjacent to the variation. A single base sequencing reaction using labeled dideoxynucleotide terminators may be used to detect the variation. The presence of a variation will result in the incorporation of the labeled terminator, whereas the absence of a variation will not result in the incorporation of the terminator. In another embodiment, a polynucleotide primer of the invention hybridizes to a variation in the LOC387715, a variation in the SYNPR gene that is correlated with AMD, or a variation in the PDGFC gene that is correlated with the occurrence of AMD. The primer, or a portion thereof, will not hybridize to LOC387715, SYNPR, or PDGFC genes that do not contain the variation that is correlated with AMD. The presence of a variation will result in primer extension, whereas the absence of a variation will not result in primer extension. The primers and/or nucleotides may further include fluorescent, radioactive, or chemical probes. A primer labeled by primer extension may be detected by measuring the intensity of the extension product, such as by gel electrophoresis, mass spectrometry, or any other method for detecting fluorescent, radioactive, or chemical labels.

In another embodiment, the presence of a variation in the LOC387715, SYNPR, or PDGFC gene that is correlated with the occurrence of AMD is detected and/or determined by ligation. In one embodiment, a polynucleotide primer of the invention hybridizes to a variation in the LOC387715, SYNPR, or PDGFC gene that is correlated with the occurrence of AMD. The primer, or a portion thereof will not hybridize to a LOC387715, SYNPR, or PDGFC gene that does not contain the variation. A second polynucleotide that hybridizes to a region of the LOC387715, SYNPR, or PDGFC gene immediately adjacent to the first primer is also provided. One, or both, of the polynucleotide primers may be fluorescently, radioactively, or chemically labeled. Ligation of the two polynucleotide primers will occur in the presence of DNA ligase if a variation in the LOC387715, SYNPR, or PDGFC gene that is correlated with the occurrence of AMD is present. Ligation may-be detected by gel electrophoresis, mass spectrometry, or by measuring the intensity of fluorescent, radioactive, or chemical labels.

In another embodiment, the presence of a variation in the LOC387715, SYNPR, or PDGFC gene that is correlated with the occurrence of AMD is detected and/or determined by single-base extension (SBE). For example, a fluorescently-labeled primer that is coupled with fluorescence resonance energy transfer (FRET) between the label of the added base and the label of the primer may be used. Typically, the method, such as that described by Chen et al., (PNAS 94:10756-61 (1997), incorporated herein by reference) uses a locus-specific polynucleotide primer labeled on the 5′ terminus with 5-carboxyfluorescein (FAM). This labeled primer is designed so that the 3′ end is immediately adjacent to the polymorphic site of interest. The labeled primer is hybridized to the locus, and single base extension of the labeled primer is performed with fluorescently labeled dideoxyribonucleotides (ddNTPs) in dye-terminator sequencing fashion, except that no deoxyribonucleotides are present. An increase in fluorescence of the added ddNTP in response to excitation at the wavelength of the labeled primer is used to infer the identity of the added nucleotide.

Methods of detecting a variation in the LOC387715, SYNPR, or PDGFC gene that is correlated with the occurrence of AMD may include amplification of a region of DNA that comprises the variation. Any method of amplification may be used. In one specific embodiment, a region of DNA comprising the variation is amplified by using polymerase chain reaction CPCR). PCR was initially described by Mullis (See e.g., U.S. Pat. Nos. 4,683,195 4,683,202, and 4,965,188, herein incorporated by reference), which describes a method for increasing the concentration of a region of DNA, in a mixture of genomic DNA, without cloning or purification. Other PCR methods may also be used to nucleic acid amplification, including but not limited to RT-PCR, quantitative PCR, real time PCR, Rapid Amplified Polymorphic DNA Analysis, Rapid Amplification of cDNA Ends (RACE), or rolling circle amplification. For example, the polynucleotide primers of the invention are combined with a DNA mixture (or any polynucleotide sequence that can be amplified with the polynucleotide primers of the invention), wherein the DNA comprises the LOC387715, SYNPR, or PDGFC gene. The mixture also includes the necessary amplification reagents (e.g., deoxyribonucleotide triphosphates, buffer, etc.) necessary for the thermal cycling reaction. According to standard PCR methods, the mixture undergoes a series of denaturation, primer annealing, and polymerase extension steps to amplify the region of DNA that comprises the variation in the LOC387715, SYNPR, or PDGFC gene. The length of the amplified region of DNA is determined by the relative positions of the primers with respect to each other, and therefore, this length is a controllable parameter. For example, hybridization of the primers may occur such that the ends of the primers proximal to the variation are separated by 1 to 10,000 base pairs (e.g., 10 base pairs (bp) 50 bp, 200 bp, 500 bp, 1,000 bp, 2,500 bp, 5,000 bp, or 10,000 bp).

Standard instrumentation known to those skilled in the art is used for the amplification and detection of amplified DNA. For example, a wide variety of instrumentation has been developed for carrying out nucleic acid amplifications, particularly PCR, e.g. Johnson et al, U.S. Pat. No. 5,038,852 (computer-controlled thermal cycler); Wittwer et al, Nucleic Acids Research, 17: 43534357 (1989)(capillary tube PCR); Hallsby, U.S. Pat. No. 5,187,084 (air-based temperature control); Garner et al, Biotechniques, 14: 112-115 (1993)high-throughput PCR in 864-well plates); Wilding et al, International application No. PCT/US93/04039 (PCR in micro-machined structures); Schnipelsky et al, European patent application No. 90301061.9 (publ. No. 0381501 A2)(disposable, single use PCR device). In certain embodiments, the invention described herein utilizes real-time PCR or other methods known in the art such as the Taqman assay.

In certain embodiments, a variant LOC387715, SYNPR, or PDGFC gene that is correlated with the occurrence of AMD in humans may be detected using single-strand conformation polymorphism analysis, which identifies base differences by alteration in electrophoretic migration of single stranded PCR products, as described in Orita et al., Proc. Nat. Acad. Sci. 86, 2766-2770 (1989). Amplified PCR products can be generated as described above, and heated or otherwise denatured, to form single stranded amplification products. Single-stranded nucleic acids may refold or form secondary structures which are partially dependent on the base sequence. The different electrophoretic mobilities of single-stranded amplification products can be related to base-sequence differences between alleles of target sequences.

In one embodiment, the amplified DNA is analyzed in conjunction with one of the detection methods described herein, such as by DNA sequencing. The amplified DNA may alternatively be analyzed by hybridization with a labeled probe, hybridization to a DNA array or microarray, by incorporation of biotinylated primers followed by avidin-enzyme conjugate detection, or by incorporation of ³²P-labeled deoxynucleotide triphosphates, such as dCTP or dATP, into the amplified segment. In a specific embodiment, the amplified DNA is analyzed by determining the length of the amplified DNA by electrophoresis or chromatography. For example, the amplified DNA is analyzed by gel electrophoresis. Methods of gel electrophoresis are well known in the art. See for example, Current Protocols in Molecular Biology, eds. Ausubel et al., John Wiley & Sons: 1992. The amplified DNA can be visualized, for example, by fluorescent or radioactive means, or with other dyes or markers that intercalate DNA. The DNA may also be transferred to a solid support such as a nitrocellulose membrane and subjected to-Southern Blotting following gel electrophoresis. In one embodiment, the DNA is exposed to ethidium bromide and visualized under ultra-violet light.

Therapeutic Nucleic Acids Encoding LOC387715, SYNPR, and PDGFC Polypeptides

In certain embodiments, the invention provides isolated and/or recombinant nucleic acids encoding a LOC387715 polypeptide, a SYNPR polypeptide, or a PDGFC polypeptide, including functional variants, disclosed herein. The subject nucleic acids may be single-stranded or double stranded. Such nucleic acids may be DNA or RNA molecules. These nucleic acids may be used, for example, in methods for malting LOC387715, SYNPR, or PDGFC polypeptides or as direct therapeutic agents (e.g., in a gene therapy approach).

The subject nucleic acids encoding LOC387715, SYNPR, or PDGFC polypeptides are further understood to include nucleic acids that are variants of sequences publicly available (e.g., through databases) and sequences referenced herein. Variant nucleotide sequences include sequences that differ by one or more nucleotide substitutions, additions or deletions, such as allelic variants; and will, therefore, include coding sequences that differ from the nucleotide sequence of the publicly available coding sequence or coding sequences referenced herein.

In certain embodiments, the invention provides isolated or recombinant nucleic acid sequences that are complementary to or are at least 80%, 85%, 90%, 95%, 97%, 98%, 99% or 100% identical to publicly available nucleic acid sequences or nucleic acid sequences referenced herein. In further embodiments, the nucleic acid sequences of the invention can be isolated, recombinant, and/or fused with a heterologous nucleotide sequence, or in a DNA library.

In other embodiments, nucleic acids of the invention also include nucleic acids that hybridize under stringent conditions to the nucleotide sequences publicly available or nucleic acid sequences referenced herein or fragments thereof. As discussed above, one of ordinary skill in the art will understand readily that appropriate stringency conditions which promote DNA hybridization can be varied. For example, one could perform the hybridization at 6.0× sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the invention provides nucleic acids which hybridize under low stringency conditions of 6×SSC at room temperature followed by a wash at 2×SSC at room temperature.

Isolated nucleic acids which differ from the nucleic acids described herein due to degeneracy in the genetic code are also within the scope of the invention. For example, a number of amino acids are designated by more than one triplet. Codons that specify the same amino acid, or synonyms (for example, CAU and CAC are synonyms for histidine) may result in “silent” variations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the_nucleotides) of the nucleic acids encoding a particular protein may exist among individuals of a given species due to natural allelic variation. Any and all such nucleotide variations and resulting amino acid polymorphisms are within the scope of this invention.

The nucleic acids and polypeptides of the invention may be produced using standard recombinant methods. For example, the recombinant nucleic acids of the invention may be operably linked to one or more regulatory nucleotide sequences in an expression construct. Regulatory nucleotide sequences will generally be appropriate to the host cell used for expression. Numerous types of appropriate expression vectors and suitable regulatory sequences are known in the art for a variety of host cells. Typically, the one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the invention. The promoters may be either naturally occurring promoters or hybrid promoters that combine elements of more than one promoter. An expression construct may be present in a cell on an episome, such as a plasmid, or the expression construct may be inserted in a chromosome. The expression vector may also contain a selectable marker gene to allow the selection of transformed host cells. Selectable marker genes are well known in the art and will vary with the host cell used.

In certain embodiments of the invention, the subject nucleic acid is provided in an expression vector comprising a nucleotide sequence encoding a LOC387715 polypeptide, a SYNPR polypeptide, or a PDGFC polypeptide and operably linked to at least one regulatory sequence. Regulatory sequences are art-recognized and are selected to direct expression of the LOC387715, SYNPR, or PDGFC polypeptide. The term regulatory sequence includes promoters, enhancers, termination sequences, preferred ribosome binding site sequences, preferred mRNA leader sequences, preferred protein processing sequences, preferred signal sequences for protein secretion, and other expression control elements. Examples of regulatory sequences are described in Goeddel; Gene Expression Technology: Methods in Enzymology; Academic Press, San Diego, Calif. (1990). For instance, any of a wide variety of expression control sequences that control the expression of a DNA sequence when operatively linked to it may be used in these vectors to express DNA sequences encoding a LOC387715, SYNPR, or PDGFC polypeptide. Such useful expression control sequences, include, for example, the early and late promoters of SV40, tet promoter, adenovirus or cytomegalovirus immediate early promoter, RSV promoters, the lac system, the trp system, the TAC or TRC system, T7 promoter whose expression is directed by T7 RNA polymerase, the major operator and promoter regions of phage lambda , the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

A recombinant nucleic acid of the invention can be produced by ligating the cloned gene, or a portion thereof, into a vector suitable for expression in either prokaryotic cells, eukaryotic cells (yeast, avian, insect or mammalian), or both. Expression vehicles for production of recombinant LOC387715, SYNPR, or PDGFC polypeptides include plasmids and other vectors. For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli.

Some mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and in transformation of host organisms are well known in the art. For other suitable expression systems for both prokaryotic and eukaryotic cells, as well as general recombinant procedures, see Molecular Cloning: A Laboratory Manual, Cold Spring Harbor Laboratory (2001). In some instances, it may be desirable to express the recombinant polypeptide by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

In one embodiment, a vector will be designed for production of a polypeptide (e.g., a LOC387715, SYNPR or PDGFC polypeptide) in CHO cells, such as a Pcmv-Script vector (Stratagene, La Jolla, Calif.), pcDNA4 vectors (Invitrogen, Carlsbad, Calif.) and pCI-neo vectors (Promega, Madison, Wisc.). In other embodiments, the vector is designed for production of a polypeptide (e.g., a LOC387715, SYNPR or PDGFC polypeptide) in prokaryotic host cells (e.g., E. coli and B. subtilis), eukaryotic host cells such as, for example, yeast cells, insect cells, myeloma cells, fibroblast 3T3 cells, monkey kidney or COS cells, mink-lung epithelial cells, human foreskin fibroblast cells, human glioblastoma cells, and teratocarcinoma cells. Alternatively, the genes may be expressed in a cell-free system such as the rabbit reticulocyte lysate system. The subject gene constructs can be used to express LOC387715, SYNPR, or PDGFC polypeptide in cells propagated in culture, e.g., to produce proteins, including fusion proteins or variant proteins, for purification.

This invention also pertains to a host cell transfected with a recombinant gene including a coding sequence for LOC387715, SYNPR, or PDGFC polypeptides. The host cell may be any prokaryotic or eukaryotic cell. For example, a LOC387715, SYNPR, or PDGFC polypeptide of the invention may be expressed in bacterial cells, such as E. coli, insect cells (e.g., using a baculovirus expression system), yeast, or mammalian cells. Other suitable host cells are known to those skilled in the art.

The present invention further pertains to methods of producing LOC387715, SYNPR, or PDGFC polypeptides. For example, a host cell transfected with an expression vector encoding a LOC387715, SYNPR, or PDGFC polypeptide can be cultured under appropriate conditions to allow expression of the LOC387715, SYNPR, or PDGFC polypeptide to occur. LOC387715, SYNPR, or PDGFC polypeptides may be secreted and isolated from a mixture of cells and medium containing the LOC387715, SYNPR, or PDGFC polypeptides. Alternatively, the polypeptide may be retained cytoplasmically or in a membrane fraction, the cells harvested and lysed and the protein isolated. A cell culture includes host cells, media and other byproducts. Suitable media for cell culture are well known in the art. The polypeptide can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the polypeptide. In a particular embodiment, the LOC387715, SYNPR, or PDGFC polypeptide is a fusion protein containing a domain which facilitates the purification of the LOC387715, SYNPR, or PDGFC polypeptide.

In another embodiment, a fusion gene coding for a purification leader sequence, such as a poly-(His)/enterokinase cleavage site sequence at the N-terminus of the desired portion of the recombinant LOC387715, SYNPR, or PDGFC-polypeptide, can allow purification of the expressed fusion protein by affinity chromatography using a Ni²⁺ metal resin. The purification leader sequence can then be subsequently removed by treatment with enterokinase to provide the purified polypeptide (e.g., see Hochuli et al., (1987) J. Chromatography 411:177; and Janknecht et al., PNAS USA 88:8972).

Techniques for making fusion genes are well known. Essentially, the joining of various DNA fragments coding for different polypeptidesequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments-can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

Antisense Polynucleotides

In certain embodiments, the invention provides polynucleotides that comprise an antisense sequence that acts through an antisense mechanism for inhibiting expression of a variant LOC387715, SYNPR, or PDGFC gene. Antisense technologies have been widely utilized to regulate gene expression (Buskirk et al., Chem Biol 11, 1157-63 (2004); and Weiss et al., Cell Mol Life Sci 55, 334-58 (1999)). As used herein, “antisense” technology refers to administration or in situ generation of molecules or their derivatives which specifically hybridize (e.g., bind) under cellular conditions, with the target nucleic acid of interest (mRNA and/or genomic DNA) encoding one or more of the target proteins so as to inhibit expression of that protein, e.g., by inhibiting transcription and/or translation, such as by steric hinderance, altering splicing, or inducing cleavage or other enzymatic inactivation of the transcript. The binding may be by conventional base pair complementarity, or, for example, in the case of binding to DNA duplexes, through specific interactions in the major groove of the double helix.

A polynucleotide that comprises an antisense sequence of the present invention can be delivered, for example, as a component of an expression plasmid which, when transcribed in the cell, produces a nucleic acid sequence that is complementary to at least a unique portion of the target nucleic acid. Alternatively, the polynucleotide that comprises an antisense sequence can be generated outside of the target cell, and which, when introduced into the target cell causes inhibition of expression by hybridizing with the target nucleic acid. Polynucleotides of the invention maybe modified so that they are resistant to endogenous nucleases, e.g. exonucleases and/or endonucleases, and are therefore stable in vivo. Examples of nucleic acid molecules for use in polynucleotides of the invention are phosphoramidate, phosphothioate and methylphosphonate analogs of DNA (see also U.S. Pat. Nos. 5,176,996; 5,264,564; and 5,256,775). General approaches to constructing polynucleotides useful in antisense technology have been reviewed, for example, by van der krol et al. (1988) Biotechniques 6:958-976; and Stein et al. (1988) Cancer Res 48:2659-2668.

Antisense approaches involve the design of polynucleotides. (either DNA or RNA) that are complementary to a target nucleic acid encoding a variant LOC387715, SYNPR, or PDGFC gene. The antisense polynucleotide may bind to an mRNA transcript and prevent translation of a protein of interest. Absolute complementarity, although preferred, is not required. In the case of double-stranded antisense polynucleotides, a single strand of the duplex DNA may thus be tested, or triplex formation may be assayed. The ability to hybridize will depend on both the degree of complementarity and the length of the antisense sequence. Generally, the longer the hybridizing nucleic acid, the more base mismatches with a target nucleic acid it may contain and still form a stable duplex (or triplex, as the case may be). One skilled in the art can ascertain a tolerable degree of mismatch by use of standard procedures to determine the melting point of the hybridized complex.

Antisense polynucleotides that are complementary to the 5′ end of an mRNA target, e.g., the 5′ untranslated sequence up to and including the AUG initiation codon, should work most efficiently at inhibiting translation of the mRNA. However, sequences complementary to the 3′ untranslated sequences of mRNAs have recently been shown to be effective at inhibiting translation of mRNAs as well (Wagner, R. 1994. Nature 372:333). Therefore, antisense polynucleotides complementary to either the 5′ or 3′ untranslated, noncoding regions of a variant LOC387715, SYNPR, OR PDGFC gene could be used in an antisense approach to inhibit translation of a variant LOC387715, SYNPR, or PDGFC mRNA. Antisense polynucleotides complementary to the 5′ untranslated region of an mRNA should include the complement of the AUG start codon. Antisense polynucleotides complementary to mRNA coding regions are less efficient inhibitors of translation but could also be used in accordance with the invention. Whether designed to hybridize to the 5′, 3′, or coding region of mRNA, antisense polynucleotides should be at least six nucleotides in length, and are preferably less that about 100 and more preferably less than about 50, 25, 17 or 10 nucleotides in length.

Regardless of the choice of target sequence, it is preferred that in vitro studies are first performed to quantitate the ability of the antisense polynucleotide to inhibit expression of a variant LOC387715, SYNPR, or PDGFC gene. It is preferred that these studies utilize controls that distinguish between antisense gene inhibition and nonspecific biological effects of antisense polynucleotide. It is also preferred that these studies compare levels of the target RNA or protein with that of an internal control RNA or protein. Additionally, it is envisioned that results obtained using the antisense polynucleotide are compared with those obtained using a control antisense polynucleotide. It is preferred that the control antisense polynucleotide is of approximately the same length as the test antisense polynucleotide and that the nucleotide sequence of the control antisense polynucleotide differs from the antisense sequence of interest no more than is necessary to prevent specific hybridization to the target sequence.

Polynucleotides of the invention, including antisense polynucleotides, can be DNA or RNA or chimeric mixtures or derivatives or modified versions thereof, single-stranded or double-stranded. Polynucleotides of the invention can be modified at the base moiety, sugar moiety, or phosphate backbone, for example, to improve stability of the molecule, hybridization, etc. Polynucleotides of the invention may include other appended groups such as peptides (e.g., for targeting host cell receptors), or agents facilitating transport across the cell membrane (see, e.g., Letsinger et al., 1989, Proc Natl Acad. Sci. USA 86:6553-6556; Lemaitre et al., 1987, Proc Natl Acad. Sci. USA 84:648-652; PCT Publication No. WO88/09810, published Dec. 15, 1988) or the blood-brain barrier (see, e.g., PCT Publication No. WO89/10134, published Apr. 25, 1988), hybridization-triggered cleavage agents. (See, e.g., Krol et al., 1988, BioTechniques 6:958-976) or intercalating agents. (See, e.g., Zon, Pharm. Res. 5:539-549 (1988)). To this end, a polynucleotide of the invention may be conjugated to another molecule, e.g., a peptide, hybridization triggered cross-linking agent, transport agent, hybridization-triggered cleavage agent, etc.

Polynucleotides of the invention, including antisense polynucleotides, may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxytriethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil; beta-D-mannosylqueosine, 5-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4:-thiouracil, 5-methyluracil, uracil-5-oxyacetic acid methyl ester, uracil-5-oxyacetic acid (v), 5-methyl-2-thiouracil, 3-(3-amino-3-N-2-carboxypropyl) uracil, (acp3)w, and 2,6-diaminopurine.

Polynucleotides of the invention may also comprise at least one modified sugar moiety selected from the group including but not limited to arabinose, 2-fluoroarabinose, xylulose, and hexose.

A polynucleotide of the invention can also contain a neutral peptide-like backbone. Such molecules are termed peptide nucleic acid (PNA)-oligomers and are described, e.g., in Perry-O'Keefe et al. (1996) Proc. Natl. Acad. Sci. USA 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, a polynucleotide of the invention comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In a further embodiment, polynucleotides of the invention, including antisense polynucleotides are -anomeric oligonucleotides. An -anomeric oligonucleotide forms specific double-stranded hybrids with complementary RNA in which, contrary to the usual -units, the strands run parallel to each other (Gautier et al., 1987, Nucl. Acids Res. 15:6625-6641). The oligonucleotide is a 2′-O-methylribonucleotide (Inoue et al., 1987, Nucl. Acids Res. 15:6131-6148), or a chimeric RNA-DNA analogue (Inoue et al., 1987, FEBS Lett. 215:327-330).

Polynucleotides of the invention, including antisense polynucleotides, may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. Nucl. Acids Res. 16:3209 (1988)), methylphosphonate oligonucleotides can be prepared by use of controlled pore glass polymer supports (Sarin et al., Proc. Natl. Acad. Sci. USA 85:7448-7451 (1988)), etc.

Antisense sequences complementary to the coding region of an mRNA sequence can be used. Alternatively, those complementary to the transcribed untranslated region and to the region comprising the initiating methionine can be used.

Antisense polynucleotides can be delivered to cells that express target genes in vivo. A number of methods have been developed for delivering nucleic acids into cells; e.g., they can be injected directly into the tissue site, or modified nucleic acids, designed to target the desired cells (e.g., antisense polynucleotides linked to peptides or antibodies that specifically bind receptors or antigens expressed on the target cell surface) can be administered systematically.

However, it may be difficult to achieve intracellular concentrations of the antisense polynucleotides sufficient to attenuate the activity of a variant LOC387715, SYNPR, or PDGFC gene or mRNA in certain instances. Therefore, another approach utilizes a recombinant DNA construct in which the antisense polynucleotide is placed under the control of a strong pol m or pol II promoter. The use of such a construct to transfect target cells in the patient will result in the transcription of sufficient amounts of antisense polynucleotides that will form complementary base pairs with the variant LOC387715, SYNPR, or PDGFC gene or mRNA and thereby attenuate the activity of LOC387715, SYNPR, or PDGFC protein. For example, a vector can be introduced in vivo such that it is taken up by a cell and directs the transcription of an antisense polynucleotide that targets a variant LOC387715, SYNPR, or PDGFC gene or mRNA. Such a vector can remain episomal or become chromosomally integrated, as long as it can be transcribed to produce the desired antisense polynucleotide. Such vectors can be constructed by recombinant DNA technology methods standard in the art. Vectors can be plasmid, viral, or others known in the art, used for replication and expression in mammalian cells. A promoter may be operably linked to the sequence encoding the antisense polynucleotide. Expression of the sequence encoding the antisense polynucleotide can be by any promoter known in the art to act in mammalian, preferably human cells. Such promoters can be inducible or constitutive. Such promoters include but are not limited to: the SV40 early promoter region (Bernoist and Chambon, Nature 290:304-310 (1981)), the promoter contained in the 3′ long terminal repeat of Rous sarcoma virus (Yamamoto et al., Cell 22:787-797 (1980)), the herpes thymidine kinase promoter (Wagner et al., Proc. Natl. Acad. Sci. USA 78:1441-1445 (1981)), the regulatory sequences of the metallothionine gene (Brinster et al, Nature 296:3942 (1982)), etc. Any type of plasmid, cosmid, YAC or viral vector can be used to prepare the recombinant DNA construct that can be introduced directly into the tissue site. Alternatively, viral vectors can be used which selectively infect the desired tissue, in which case administration may be accomplished by another route (e.g., systematically).

RNAi Constructs—siRNAs and miRNAs

RNA interference (RNAi) is a phenomenon describing double-stranded (ds)RNA-dependent gene specific posttranscriptional silencing. The present invention provides a polynucleotide comprising an RNAi sequence that acts through an RNAi or miRNA mechanism to attenuate expression of a variant LOC387715, SYNPR, or PDGFC gene. For instance, a polynucleotide of the invention may comprise a miRNA or siRNA sequence that attenuates or inhibits expression of a variant LOC387715, SYNPR, or PDGFC gene. In one embodiment, the miRNA or siRNA sequence is between about 19 nucleotides and about 75 nucleotides in length, or preferably, between about 25 base pairs and about 35 base pairs in-length. In certain embodiments, the polynucleotide is a hairpin loop or stem-loop that may be processed by RNAse enzymes (e.g., Drosha and Dicer).

An RNAi construct contains a nucleotide sequence that hybridizes under physiologic conditions of the cell to the nucleotide sequence of at least a portion of the mRNA transcript for a variant LOC387715, SYNPR, or PDGFC gene. The double-stranded RNA need only be sufficiently similar to natural RNA that it has the ability to mediate RNAi. The number of tolerated nucleotide mismatches between the target sequence and the RNAi construct sequence is no more than 1 in 5 basepairs, or 1 in 10 basepairs, or 1 in 20 basepairs, or 1 in 50 basepairs. It is primarily important the that RNAi construct is able to specifically target a variant LOC387715, SYNPR, or PDGFC gene. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing).

Production of polynucleotides comprising RNAi sequences can be carried out by a variety of methods. For example, polynucleotides comprising RNAi sequences can be produced by chemical synthetic methods or by recombinant nucleic acid techniques. Endogenous RNA polymerase of the treated cell may mediate transcription in vivo, or cloned RNA polymerase can be used for transcription in vitro. Polynucleotides of the invention, including wildtype or antisense polynucleotides, or those that modulate target gene activity by RNAi mechanisms, may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of a nitrogen or sulfur heteroatom. Modifications in RNA structure may be tailored to allow specific genetic inhibition while avoiding a general response to dsRNA. Likewise, bases may be modified to block the activity of adenosine deaminase. Polynucleotides of the invention may be produced enzymatically or by partial/total organic synthesis, any modified ribonucleotide can be introduced by in vitro enzymatic or organic synthesis.

Methods of chemically modifying RNA molecules can be adapted for modifying RNAi constructs (see, for example, Heidenreich et al. (1997) Nucleic Acids Res, 25:776-780; Wilson et al. (1994) J Mol Recog 7:89-98; Chen et al. (1995) Nucleic Acids Res 23:2661-2668; Hirschbein et al. (1997) Antisense Nucleic Acid Drug Dev 7:55-61). Merely to illustrate, the backbone of an RNAi construct can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, a-configuration).

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

In certain embodiments, the subject RNAi constructs are “siRNAs.” These nucleic acids are between about 19-35 nucleotides in length, and even more preferably 21-23 nucleotides in length, e.g., corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the protein complex or translation is inhibited. In a particular embodiment, the 21-23 nucleotides siRNA molecules comprise a 3′ hydroxyl group.

In other embodiments, the subject RNAi constructs are “miRNAs.” microRNAs (miRNAs) are small non-coding RNAs that direct post transcriptional regulation of gene expression through interaction with homologous mRNAs. miRNAs control the expression of genes by binding to complementary sites in target mRNAs from protein coding genes. miRNAs are similar to siRNAs. miRNAs are processed by nucleolytic cleavage from larger double-stranded precursor molecules. These precursor molecules are often hairpin structures of about 70 nucleotides in length, with 25 or more nucleotides that are base-paired in the hairpin. The RNAse EU-like enzymes Drosha and Dicer (which may also be used in siRNA processing) cleave the miRNA precursor to produce an miRNA. The processed miRNA is single-stranded and incorporates into a protein complex, termed RISC or miRNP. This RNA-protein complex targets a complementary mRNA. miRNAs inhibit translation or direct cleavage of target mRNAs. (Brennecke et al., Genome Biology 4:228 (2003); Kim et al., Mol. Cells. 19:1-15 (2005).

In certain embodiments, miRNA and siRNA constructs can be generated by processing of longer double-stranded RNAs, for example, in the presence of the enzymes Dicer or Drosha. Dicer and Drosha are RNAse III-like nucleases that specifically cleave dsRNA. Dicer has a distinctive structure which includes a helicase domain and dual RNAse III motifs. Dicer also contains a region of homology to the RDE1/QDE2/ARGONAUTE family, which have been genetically linked to RNAi in lower eukaryotes. Indeed, activation of, or overexpression of Dicer may be sufficient in many cases to permit RNA interference in otherwise non-receptive cells, such as cultured eukaryotic cells, or mammalian (non-oocytic) cells in culture or in whole organisms. Methods and compositions employing Dicer, as well as other RNAi enzymes, are described in U.S. Pat. App. Publication No. 2004/0086884.

In one embodiment, the Drosophila in vitro system is used. In this embodiment, a polynucleotide comprising an RNAi sequence or an. RNAi precursor is combined with a soluble extract derived from Drosophila embryo, thereby producing a combination. The combination is maintained under conditions in which the dsRNA is processed to RNA molecules of about 21 to about 23 nucleotides.

The miRNA and siRNA molecules can be purified using a number of techniques known to those of skill in the art. For example, gel electrophoresis can be used to purify such molecules. Alternatively, non-denaturing methods, such as non-denaturing column chromatography, can be used to purify the siRNA and miRNA molecules. In addition, chromatography (e.g., size exclusion chromatography), glycerol gradient centrifugation, affinity purification with antibody can be used to purify siRNAs and miRNAs.

In certain embodiments, at least one strand of the siRNA sequence of an effector domain has a 3′ overhang from about 1 to about 6 nucleotides in length, or from 2 to 4 nucleotides in length. In other embodiments, the 3′ overhangs are 1-3 nucleotides in length. In certain embodiments, one strand has a 3′ overhang and the other strand is either blunt-ended or also has an overhang. The length of the overhangs may be the same or different for each strand. In order to further enhance the stability of the siRNA sequence, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e-g., substitution of uridine nucleotide 3′ overhangs by 2′-deoxythyinidine is tolerated and does not affect the efficiency of RNAi. The absence of a 2′ hydroxyl significantly enhances the nuclease resistance of the overhang in tissue culture medium and may be beneficial in vivo.

In certain embodiments, a polynucleotide of the invention that comprises an RNAi sequence or an RNAi precursor is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA 2002, 8:842-50; Yu et al., Proc Natl Acad Sci USA, 2002, 99:6047-52). Preferably, such hairpin RNAs are engineered in cells or in an animal to ensure continuous and stable suppression of a desired gene. It is known in the art that miRNAs and siRNAs can be produced by processing a hairpin RNA in the cell.

In yet other embodiments, a plasmid is used to deliver the double-stranded RNA, e.g., as a transcriptional product. After the coding sequence is transcribed, the complementary RNA transcripts base-pair to form the double-stranded RNA.

Antibodies

Another aspect of the invention pertains to antibodies. In one embodiment, an antibody that is specifically reactive with a variant LOC387715, SYNPR, or PDGFC polypeptide may be used to detect the presence of a variant LOC387715, SYNPR, or PDGFC polypeptide or to inhibit activity of a variant LOC387715, SYNPR, or PDGFC polypeptide. For example, by using immunogens derived from a variant LOC387715, SYNPR, or PDGFC peptide, anti-protein/anti-peptide antisera or monoclonal antibodies can be made by standard protocols (see, for example, Antibodies: A Laboratory Manual ed. by Harlow and Lane (Cold Spring Harbor Press 1988). A mammal, such as a mouse, a hamster or rabbit can be immunized with an immunogenic form of the variant LOC387715, SYNPR, or PDGFC peptide, an antigenic fragment which is capable of eliciting an antibody response, or a fusion protein. In a particular embodiment, the inoculated mouse does not express endogenous LOC387715, SYNPR, or PGDFC, thus facilitating the isolation of antibodies that would otherwise be eliminated as anti-self antibodies. Techniques for conferring immunogenicity on a protein or peptide include conjugation to carriers or other techniques well known in the art. An immunogenic portion of a variant LOC387715, SYNPR, or PDGFC peptide can be administered in the presence of adjuvant. The progress of immunization can be monitored by detection of antibody titers in plasma or serum. Standard ELISA or other immunoassays can be used with the immunogen as antigen to assess the levels of antibodies.

Following immunization of an animal with an antigenic preparation of a variant LOC387715, SYNPR, or PDGFC polypeptide, antisera can be obtained and, if desired, polyclonal antibodies can be isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with a variant LOC387715, SYNPR, or PDGFC polypeptide and monoclonal antibodies isolated from a culture comprising such hybridoma cells.

The term “antibody” as used herein is intended to include fragments thereof which are also specifically reactive with a variant LOC387715, SYNPR, or PDGFC polypeptide . Antibodies can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to produce Fab fragments. The antibody of the present invention is further intended to include bispecific, single-chain, and chimeric and humanized molecules having affinity for a variant LOC387715, SYNPR, or PDGFC polypeptide conferred by at least one CDR region of the antibody. In preferred embodiments, the antibody further comprises a label attached thereto and able to be detected (e.g., the label can be a radioisotope, fluorescent compound, enzyme or enzyme co-factor).

In certain embodiments, an antibody of the invention is a monoclonal antibody, and in certain embodiments, the invention makes available methods for generating novel antibodies that bind specifically to variant LOC387715, SYNPR, or PDGFC polypeptide s. For example, a method for generating a monoclonal antibody that binds specifically to a variant LOC387715, SYNPR, or PDGFC polypeptide may comprise administering to a mouse an amount of an immunogenic composition comprising the LOC387715, SYNPR, or PDGFC polypeptide effective to stimulate a detectable immune response, obtaining antibody-producing cells (e.g., cells from the spleen) from the mouse and fusing the antibody-producing cells with myeloma cells to obtain antibody-producing hybridomas, and testing the antibody-producing hybridomas to identify a hybridoma that produces a monocolonal antibody that binds specifically to the variant LOC387715, SYNPR, or PDGFC polypeptide. Once obtained, a hybridoma can be propagated in a cell culture, optionally in culture conditions where the hybridoma-derived cells produce the monoclonal antibody that binds specifically to the LOC387715, SYNPR, or PDGFC polypeptide. The monoclonal antibody may be purified from the cell culture.

The term “specifically reactive with” as used in reference to an antibody is intended to mean, as is generally understood in the art, that the antibody is sufficiently selective between the antigen of interest (e.g., a variant LOC387715, SYNPR, or PDGFC polypeptide ) and other antigens that are not of interest that the antibody is useful for, at minimum, detecting the presence of the antigen of interest in a particular type of biological sample. In certain methods employing the antibody, such as therapeutic applications, a higher degree of specificity in binding may be desirable. Monoclonal antibodies generally have a greater tendency (as compared to polyclonal antibodies) to discriminate effectively between the desired antigens and cross-reacting polypeptides. One characteristic that influences the specificity of an antibody:antigen interaction is the affinity of the antibody for the antigen. Although the desired specificity may be reached with a range of different affinities, generally preferred antibodies will have an affinity (a dissociation constant) of about 10⁻⁶, 10⁻⁷, 10⁻⁸, 10⁻⁹ or less.

Screening Assays

The present invention relates to the use of LOC387715, SYNPR, or PDGFC polypeptides to identify compounds (agents) which are agonists or antagonists of LOC387715, SYNPR, or PDGFC polypeptides. Compounds identified through this screening can be tested in cells of the eye, (e.g., epithelial and endothelial cells) as well as other tissues (e.g., muscle and/or neurons) to assess their ability to modulate LOC387715, SYNPR, or PDGFCactivity in vivo or in vitro. In certain aspects, compounds identified through this screening modulate the formation of drusen deposits. Optionally, these compounds can further be tested in animal models to assess their ability to modulate LOC387715, SYNPR, or PDGFC activity in vivo.

There are numerous approaches to screening for therapeutic agents that target LOC387715, SYNPR, or PDGFC polypeptides. In certain embodiments, high-throughput screening of compounds can be carried out to identify agents that affect activity of LOC387715, SYNPR, or PDGFC polypeptides. A variety of assay formats will suffice and, in light of the present disclosure, those not expressly described herein will nevertheless be comprehended by one of ordinary skill in the art. As described herein, the test compounds (agents) of the invention may be created by any combinatorial chemical method. Alternatively, the subject compounds may be naturally occurring biomolecules synthesized in vivo or in vitro. Compounds (agents) to be tested for their ability to act as modulators of LOC387715, SYNPR, or PDGFCactivity can be produced, for example, by bacteria, yeast, plants or other organisms (e.g., natural products), produced chemically (e.g., small molecules, including peptidomimetics), or produced recombinantly. Test compounds contemplated by the present invention include non-peptidyl organic molecules, peptides, polypeptides, peptidomimetics, sugars, hormones, and nucleic acid molecules.

The test compounds of the invention can be provided as single, discrete entities, or provided in libraries of greater complexity, such as made by combinatorial chemistry. These libraries can comprise, for example, alcohols, alkyl halides, amines, amides, esters, aldehydes, ethers and other classes of organic compounds. Presentation of test compounds to the test system can be in either an isolated form or as mixtures of compounds, especially in initial screening steps. Optionally, the compounds may be optionally derivatized with other compounds and have derivatizing groups that facilitate isolation of the compounds. Non-limiting examples of derivatizing groups include biotin, fluorescein, digoxygenin, green fluorescent protein, isotopes, polyhistidine, magnetic beads, glutathione S transferase (GST), photoactivatible crosslinkers or any combinations thereof.

Pharmaceutical Compositions

The methods and compositions described herein for treating a subject suffering from AMD may be used for the prophylactic treatment of individuals who have been diagnosed or predicted to be at risk for developing AMD. In this case, the composition is administered in an amount and dose that is sufficient to delay, slow, or prevent the onset of AMD or related symptoms. Alternatively, the methods and compositions described herein may be used for the therapeutic treatment of individuals who suffer from AMD. In this case, the composition is administered in an amount and dose that is sufficient to delay or slow the progression of the condition, totally or partially, or in an amount and dose that is sufficient to reverse the condition to the point of eliminating the disorder. It is understood that an effective amount of a composition for treating a subject who has been diagnosed or predicted to be at risk for developing AMD is a dose or amount that is in sufficient quantities to treat a subject or to treat the disorder itself.

In certain embodiments, compounds of the present invention (e.g., an isolated or recombinantly produced nucleic acid molecule coding for a LOC387715, SYNPR, or PDGFC polypeptide or an isolated or recombinantly produced LOC387715, SYNPR, or PDGFC polypeptide) are formulated with a pharmaceutically acceptable carrier. For example, a LOC387715, SYNPR, or PDGFC polypeptide or a nucleic acid molecule coding for a LOC387715, SYNPR, or PDGFC polypeptide can be administered alone or as a component of a pharmaceutical formulation (therapeutic composition). The subject compounds may be formulated for administration in any convenient way for use in human medicine.

In certain embodiments, the therapeutic methods of the invention include administering the composition topically, systemically, or locally. For example, therapeutic compositions of the invention may be formulated for administration by, for example, injection (e.g., intravenously, subcutaneously, or intramuscularly), inhalation or insufflation (either through the mouth or the nose) or oral, buccal, sublingual, transdermal, nasal, or parenteral administration. The compositions described herein may be formulated as part of an implant or device. When administered, the therapeutic composition for use in this invention is in a pyrogen-free, physiologically acceptable form. Further, the composition may be encapsulated or injected in a viscous form for delivery to the site where the target cells are present, such as to the cells of the eye. Techniques and formulations generally may be found in Remington's Pharmaceutical Sciences, Meade Publishing Co., Easton, Pa. In addition to LOC387715, SYNPR, or PDGFC polypeptides or nucleic acid molecules coding for LOC387715, SYNPR, or PDGFC polypeptides, therapeutically useful agents may optionally be included in any of the compositions as described above. Furthermore, therapeutically useful agents may, alternatively or additionally, be administered simultaneously or sequentially with LOC387715, SYNPR, OR PDGFC polypeptides or nucleic acid molecules coding for LOC387715, SYNPR, or PDGFC polypeptides according to the methods of the invention.

In certain embodiments, compositions of the invention can be administered orally, e.g., in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an agent as an active ingredient. An agent may also be administered as a bolus, electuary or paste.

In solid dosage forms for oral administration (capsules, tablets, pills, dragees, powders, granules, and the like), one or more therapeutic compounds of the present invention may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay, (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches, and inhalants. The active compound may be mixed under sterile conditions with a pharmaceutically acceptable carrier, and with any preservatives, buffers, or propellants which may be required. The ointments, pastes, creams and gels may contain, in addition to a subject compound of the invention (e.g., an isolated or recombinantly produced nucleic acid molecule coding for a LOC387715, SYNPR, or PDGFC polypeptide or an isolated or recombinantly produced LOC387715, SYNPR or PDGFC polypeptide ), excipients, such as animal and vegetable fats, oils; waxes, paraffins, starch, tragacanth, cellulose derivatives, polyethylene glycols, silicones, bentonites, silicic acid, talc and zinc oxide, or mixtures thereof.

Powders and sprays can contain, in addition to a subject compound, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

The dosage regimen will be determined for an individual, taking into consideration, for example, various factors which modify the action of the subject compounds of the invention, the severity or stage of AMD, route of administration, and characteristics unique to the individual, such as age, weight, and size. A person of ordinary skill in the art is able to determine the required dosage to treat the subject. In one embodiment, the dosage can range from about 1.0 ng/kg to about 100 mg/kg body weight of the subject. Based upon the composition, the dose can be delivered continuously, or at periodic intervals. For example, on one or more separate occasions. Desired time intervals of multiple doses of a particular composition can be determined without undue experimentation by one skilled in the art. For example, the compound may be delivered hourly, daily, weekly, monthly, yearly (e.g. in a time release form) or as a one time delivery.

In certain embodiments, pharmaceutical compositions suitable for parenteral administration may comprise a LOC387715, SYNPR, or PDGFC polypeptide or a nucleic acid molecule coding for a LOC387715, SYNPR, or PDGFC polypeptide in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecitin, by the maintenance of the required particle size in the case of dispersions, and by the use of surfactants.

The compositions of the invention may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

In certain embodiments, the present invention also provides gene therapy for the in vivo production of LOC387715, SYNPR, or PDGFC polypeptides. Such therapy would achieve its therapeutic effect by introduction of LOC387715, SYNPR, or PDGFCpolynucleotide sequences into cells or tissues. that are deficient for normal LOC387715, SYNPR, or PDGFC function. Delivery of LOC387715, SYNPR, or PDGFCpolynucleotide sequences can be achieved using a recombinant expression vector such as a chimeric virus or a colloidal dispersion system. Targeted liposomes may also be used for the therapeutic delivery of LOC387715, SYNPR, or PDGFCpolynucleotide sequences.

Various viral vectors which can be utilized for gene therapy as taught herein include adenovirus, herpes virus, vaccinia, or an RNA virus such as a retrovirus. A retroviral vector may be a derivative of a murine or avian retrovirus. Examples of retroviral vectors in which a single foreign gene can be inserted include, but are not limited to: Moloney murine leukemia virus (MoMuLV), Harvey murine sarcoma virus HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated. Retroviral vectors can be made target-specific by attaching, for example, a sugar, a glycolipid, or a protein. Preferred targeting is accomplished by using an antibody. Those of skill in the art will recognize that specific polynucleotide sequences can be inserted into the retroviral genome or attached to a viral envelope to allow target specific delivery of the retroviral vector containing the LOC387715, SYNPR, or PDGFC polynucleotide. In one preferred embodiment, the vector is targeted to cells or tissues of the eye.

Alternatively, tissue culture cells can be directly transfected with plasmids encoding the retroviral structural genes gag, pol and env, by conventional calcium phosphate transfection. These cells are then transfected with the vector plasmid containing the genes of interest. The resulting cells release the retroviral vector into the culture medium.

Another targeted delivery system for LOC387715, SYNPR, or PDGFC polynucleotides is a colloidal dispersion system. Colloidal dispersion systems include macromolecule complexes, nanocapsules, microspheres, beads, and lipid-based systems including oil-in-water emulsions, micelles, mixed micelles, and liposomes. The preferred colloidal system of this invention is a liposome. Liposomes are artificial membrane vesicles which are useful as delivery vehicles in vitro and in vivo. RNA, DNA and intact virions can be encapsulated within the aqueous interior and be delivered to cells in a biologically active form (see e.g., Fraley, et al., Trends Biochem. Sci., 6:77, 1981). Methods for efficient gene transfer using a liposome vehicle, are known in the art, see e.g., Mannino, et al., Biotechniques, 6:682, 1988. The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Illustrative phospholipids include egg phosphatidylcholine dipalmitoylphosphatidylcholine, and distearoylphosphatidylcholine. The targeting of liposomes is also possible based on, for example, organ-specificity, cell-specificity, and organelle-specificity and is known in the art.

A person of ordinary skill in the art is able to determine the required amount to treat the subject. It is understood that the dosage regimen will be determined for an individual, taking into consideration, for example, various factors which modify the action of the subject compounds of the invention, the severity or stage of AMD, route of administration, and characteristics unique to the individual, such as age, weight, and size. A person of ordinary skill in the art is able to determine the required dosage to treat the subject. In one embodiment, the dosage can range from about 1.0 ng/kg to about 100 mg/kg body weight of the subject. The dose can be delivered continuously, or at periodic intervals. For example, on one or more separate occasions. Desired time intervals of multiple doses of a particular composition can be determined without undue experimentation by one skilled in the art. For example, the compound may be delivered hourly, daily, weekly, monthly, yearly (e.g. in a time release form) or as a one time delivery. As used herein, the term subject or individual means any animal capable of becoming afflicted with AMD. The subjects include, but are not limited to, human beings, primates, horses, birds, cows, pigs, dogs, cats, mice, rats, guinea pigs, ferrets, and rabbits.

Samples used in the methods described herein may comprise cells from the eye, ear, nose, teeth, tongue, epidermis, epithelium, blood, tears, saliva, mucus, urinary tract, urine, muscle, cartilage, skin, or any other tissue or bodily fluid from which sufficient DNA or RNA can be obtained.

The sample should be sufficiently processed to render the DNA or RNA that is present available for assaying in the methods described herein. For example, samples may be processed such that DNA from the sample is available for amplification or for hybridization to another polynucleotide. The processed samples may be crude lysates where available DNA or RNA is not purified from other cellular material. Alternatively, samples may be processed to isolate the available DNA or RNA from one or more contaminants that are present in its natural source. Samples may be processed by any means known in the art that renders DNA or RNA available for assaying in the methods described herein. Methods for processing samples may include, without limitation, mechanical, chemical, or molecular means of lysing and/or purifying cells and cell lysates. Processing methods may include, for example, ion-exchange chromatography, size exclusion chromatography, affinity chromatography, hydrophobic interaction chromatography, gel filtration chromatography, ultrafiltration, electrophoresis, and immunoaffinity purification with antibodies specific for particular epitopes of the polypeptide

Kits

Also provided herein are kits, e.g., kits for therapeutic purposes or kits for detecting a variant LOC387715, SYNPR, or PDGFC gene in a sample from an individual. In one embodiment, a kit comprises at least one container means having disposed therein a premeasured dose of a polynucleotide probe that hybridizes, under stringent conditions, to a variation in the LOC387715 gene, a variation in the SYNPR gene, or a variation in the PDGFC gene that is correlated with the occurrence of AMD in humans. In another embodiment, a kit comprises at least one container means having disposed therein a premeasured dose of a polynucleotide primer that hybridizes, under stringent conditions, adjacent to one side of a variation in the LOC387715 gene, a variation in the SYNPR gene, or a variation in the PDGFC gene that is correlated with the occurrence of AMD in humans. In a further embodiment, a second polynucleotide primer that hybridizes, under stringent conditions, to the other side of a variation in the LOC387715 gene, a variation in the SYNPR gene, or a variation in the PDGFC gene that is correlated with the occurrence of AMD in humans is provided in a premeasured dose. Kits may include one or more than one probe or primer, such as one or more probe or primer that hybridizes to a variation in LOC387715; one or more probe or primer that hybridizes to SYNPR; and one or more probe or primer that hybridizes to PDGFC. They may additionally comprise one or more probe or primer that hybridizes to a variation in CFH that is correlated with AMD and/or one or more probe or primer that hybridizes to one or more of the corresponding genes that do not comprise the variation of interest (e.g., control or reference genes). Kits further comprise a label and/or instructions for the use of the therapeutic or diagnostic kit in the detection of LOC387715, SYNPR, or PDGFC in a sample. Kits may also include packaging material such as, but not limited to, ice, dry ice, styrofoam, foam, plastic, cellophane, shrink wrap, bubble wrap, paper, cardboard, starch peanuts, twist ties, metal clips, metal cans, drierite, glass, and rubber (see products available from www.papermart.com. for examples of packaging material).

The practice of the present methods will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning: A Laboratory Manual, Cold Spring Harbor Laboratory (2001); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. K Weir and C. C. Blackwell, eds., 1986); Manipulating the Mouse Embryo, (Cold Spring Harbor Laboratory Press, Cold Spring Harbor, N.Y., 1986).

### EXEMPLIFICATION

Analysis of genome-wide SNP genotyping data from 96 cases with AMD and 50 controls without AMD was carried out to identify both single associations at LOC387715 and other loci that appear to interact.

The following methods and materials have been used in the work described herein.
Haplotype analysis. To impute haplotypes at 10q26, Applicants used SNPHAP version 1.3 with default parameters. Haplotype inference is subject to errors and so Applicants also imputed haplotypes over the same region using PHASE version 2.1.1. The two programs use different approaches to estimate haplotypes, and would therefore presumably be subject to different errors. Both programs produced identical results on the AREDS data, suggesting accurate haplotype estimations.
Test for interactions. A proper analysis of the interactions from high dimensional data that contain more than 100,000 SNPs may be performed at two stages: selecting markers with the statistically significant joint effects, and then modeling the selected markers to quantify the extent of the effects (10). What follows is the first-stage procedure; the conventional (logistic) regression analysis can be employed at the second stage.

A simple two-locus test of association would involve comparing the frequency of the 9 two locus genotypes between cases and controls. Significant differences could indicate epistasis or major locus effects. To specifically focus on interactions, Applicants first grouped two-locus genotypes into two or three classes (numbered 1, 2 or 1, 2, 3, respectively) and tested for differences in class frequencies between cases and controls (Table 1). They used four different classification schemes (Type I-IV) inspired by Cockerham's partitioning of epistatic variance (11). For each scheme, A and a represent the alleles at locus 1 (always based on the LOC387715 haplotypes in Applicants' data, as described below) and B and b represent the alleles at locus 2 (always a SNP in Applicants data). The classes are defined in Table 1.

For each pair of loci to be tested, four independent tests were performed for type I, II, III, and IV interactions. For each test, the two-locus genotype for each individual was recoded into 2 or 3 categories using one of the tables in Table 1. Genotypic class counts were then compared between cases and controls. Statistical significance was assessed using a Pearson λ² test with two (types I-E) or one (type IV) degrees of freedom. Multiple tests were corrected using a Bonferroni correction for 116204 SNPs*4=464816 total tests.

Expression analysis. Human retina, placenta, kidney, and liver samples were obtained from National Disease Research Interchange (NDRI). Native human RPE and cultured human RPE were kindly provided by Dr. Bret Hughes and Dr. Piyoush Kothary, respectively.

Total RNA was isolated using TRIZOL (Invitrogen). First-strand cDNA was generated from 2.5 mg of total RNA by priming with oligo-dT followed by reverse transcription (www.invitrogen.com). Primers spanning introns were designed using Primer3 software (http://frodo.wi.mit.edu/cgi-bin/primer3/primer3_www.cgi) to avoid amplification from genomic DNA present in total RNA preparations. PCR reactions were set up using standard conditions. The expected product sizes are 300 bp for PDFGC, 250 bp for SYNPR, and 375 bp for LOC387715. The primers used were as follows:

| | |
|---|---|
| PDGFC-F | |
| 5′-GCTGCACACCTCGTAACTTCT-3′ | (SEQ ID NO. 1) |
| PDGFC-R | |
| 5′-GATGCGGCTATCCTCCTGT-3′ | (SEQ ID NO. 2) |
| SYNPR-F | |
| 5′-AAACACTTCTGTGGTCTTTGGA-3′ | (SEQ ID NO. 3) |
| SYNPR-R | |
| 5′-AGTGGGGCCAGTAGGCTGT-3′ | (SEQ ID NO. 4) |
| LOC387715-F | |
| 5′-TCCCAGCTGCTAAAATCCAC-3′ | (SEQ ID NO. 5) |
| LOC387715-R | |
| 5′-GCTGCACAGAGCAGAAGATG-3′ | (SEQ ID NO. 6) |

Tissue preparation. Normal donor eyes were fixed in 4% paraformaldehyde (EM Grade, Polysciences, Warrington, Pa.) in in phosphate buffer saline (PBS) for 6 hours, cryo-protected, and embedded in optimal cutting temperature compound (OCT; Miles Laboratory, Elkdhart, Ind.). Frozen retinas sections were cut at 8 to 10 μm with a cryostat (Leica microsystem, Bannockburn, Ill.) and placed on slides (Superfrost/Plus; Fisher Scientific, Fair Lawn, N.J.). All human eyes were obtained with the informed consent of the donors, and the research with human eyes was performed in accordance with the tenets of the Declaration of Helsinki and the institutional review board (IRB).
Immunofluorescence Microscopy. The retina sections were blocked for 30 minutes with 5%-normal goat serum (Jackson Immunoresearch, West Grove, Pa.) diluted in IC buffer (PBS, containing 0.2% Tween-20, 0.1% sodium azide) and incubated for 1 hour at room temperature with a rabbit anti-rat synaptoporin antiserum (SYSY, Gottingen, Germany) diluted 1:50 in staining buffer (IC buffer plus 1% normal goat serum). Sections were washed 3 times in IC buffer and incubated for 1 hour with the nuclear dye 4′,6′-diamino-2-phenylindole (DAPI; 1 μg/mL) and Alexa-488 Goat anti-rabbit antibodies (Molecular Probes, Eugene, Oreg.) diluted 1:250 in staining buffer. After repeated washing with IC buffer, sections were covered in mounting medium (Gel Mount; Biomeda, Foster City, Calif.) and coverslipped. For the control, the same concentration of anti-synaptoporin antibody was preincubated for 1 hour with the synaptoporin control peptide (SYSY, Gottingen, Germany). The pretreated antibodies were then used to stain tissue sections as just described. Specimens were analyzed on a laser scanning confocal microscope (model SP2; Leica Microsystems, Exton, Pa.). Immunolabeled and negative control sections were imaged under identical scanning conditions. Images were processed with Photoshop (Adobe Systems, San Jose, Calif.)

### Results

SNP rs10490924 by itself was barely statistically significant in the AREDS dataset (both allelic and genotypic nominal p-values are 0.04; Table 2), in part due to a lower frequency of the risk allele in the case group, compared to the two published reports. This frequency difference might be due to different definitions of AMD in these studies. In the study described herein, individuals were required to have drusen greater than 125 μm in size to be cases (1), whereas in other studies, pigmentary changes, neovascularization, or geographic atrophy were sufficient for a diagnosis of AMD (8, 9). These observations and additional analyses of Applicants' data indicated the existence of other variants acting in concert with SNP rs10490924 to jointly influence the disease risk. To explore this further, a set of four SNPs surrounding rs10490924 were defined, and showed evidence of ancestral recombination with flanking markers using the four-gamete test (FGT). These four SNPs cover approximately 500 nucleotides. Four of the 16 possible haplotypes accounted for all of the chromosomes in the sample. Two haplotypes were “risk” haplotypes (N2 and N3), while the other two were “not-risk” haplotypes (N1 and N4; Table 3). The two risk haplotypes are tagged by SNPs rs2736911 and rs10490924. The difference between “risk” and “not-risk” haplotype frequencies is statistically significant in cases versus controls (Table 2).

The imputed haplotypes for each individual were used to define a new “SNP” For this SNP, an allele “A” means haplotype N1 or N4, and an allele “B” means haplotype N2 or N3. An individual's genotype at this “SNP” was assigned based on the haplotypes of the two chromosomes in that individual. A two-way interaction test was performed to examine differential interactions in cases and controls between this derived “SNP” (here called 10q26Hap) and all other SNPs in the genome-wide study. Using a strict Bonferroni threshold p<0.05/(4×10⁵), Applicants obtained two significant results (Pearson χ² test; contingency tables in Table 4), which were later replicated in another population cohort (see below). No significant interaction was found with two SNPs in CH Applicants previously found to be associated with AMD. For Y402H, the best interaction had an uncorrected P-value of 0.093. For rs380390, the best interaction had an uncorrected P of 0.11. This implies the interactions significant at the Bonferroni threshold may be real and further investigation is warranted.

One of these interactions is between SNP rs10510899 on chromosome 3p14.2 and 10q26Hap; the second is between SNP rs997955 on chromosome 4q32.1 and 10q26Hap. SNPs rs10510899 and rs997955 are in introns of synaptoporin (SYNPR) and platelet-derived growth factor C(PDGFC), respectively. Individually, these SNPs did not exhibit single-locus association with AMD in the present study. Notably, these two SNPs are located within two of the top six ranked regions that were identified in a recent meta-analysis of AMD linkage studies (2). Even though the entire genome was scanned in this study for interactions in a hypothesis-free manner, the only two SNPs significant at the Bonferroni threshold are located in regions that Applicants would have hypothesized were involved in AMD based on previous linkage studies. In one study, these four genes (CFH, LOC387715, SYNPR, and PDGFC) were within four of the major linkage peaks and an interaction between them has been implied (12).

With the data presented here, an overall genetic risk for AMD was estimated. The number of histidine alleles in CFH at position 402 and the number of risk haplotypes at 10q26 in a given individual were summed. If persons at risk were defined as those having a sum of at least two, 82% of the cases would be classified to be at risk, but 48% of the controls would also be classified to be at risk. Instead, the risk was defined based on the genotypes of the five SNPs at the four distinct loci. The overall genetic risk was the sum of three independent risk factors: CFH, the interaction of 10q26Hap with rs10510899 in SYNPR, and the interaction of 10q26Hap with rs997955 in PDGFC. The three possible genotypes or genotypic classes for each risk factor are given a score ranging from 0 (least risk) to 2 (most risk). The sum of these scores is taken as a measure of overall risk. Individuals with an overall score of 3 or more were considered to be “at risk,” while everyone with a score of 2 or less was “not at risk” (Table 2). With this classification, 81% of the cases are at risk compared with 36% of the controls. A population attributable risk (PAR) for the effect of this genetic network (Table 2) was estimated to be of 71%.

Because these interactions are largely data derived, there is the possibility of false positives due to over-fitting of Applicants' data to the statistical models for genetic interactions. The best way to assess whether this is a chance or real association is to replicate the genotyped SNPs in a second, independent cohort of case and control individuals. DNA was obtained from patients and controls collected at the University of Michigan (6). As in the initial AREDS cohort, all individuals were of European descent to reduce the possibility of false positives due to population stratification. In this independent group of cases and controls, SNP rs10490924 is strongly and significantly associated with AMD risk (Table 2). The risk allele frequency observed for the single SNP in the cases is similar to that of the two previous studies but different from that observed in the AREDS sample. This difference could occur because the Michigan case cohort also included individuals with geographic atrophy and/or neovascularization who did not have large drusen (>125 μm diameter), because of subtle differences in the genetic background of patients in the two studies, or because the Michigan sample was enriched for familial cases.

Further analysis shows that the two classes of haplotypes at 10q26 are significantly associated with AMD, as are the two interactions identified in the AREDS cohort (Table 2; contingency tables in Table 4). In some cases the odds ratios for the same risk factor are quite different in the two cohorts. This is likely due to the small sample size of the AREDS cohort, and is reflected in the large confidence intervals for this cohort. Additional studies using large cohorts will be needed to precisely compute the odds ratios for these risk factors. However, as all of the reported interactions are statistically significant in the relatively small AREDS cohort and independently replicated in the Michigan cohort, Applicants concluded that these are biologically important interactions. Using the same definition of risk (at least 3 risk factors), Applicants observed that 63% of the cases were at risk compared to 32% of controls (Table 2). For the Michigan sample, the estimated PAR is was 55%. The extreme phenotypes in the AREDS study may explain the discrepancy in PARs between the two samples.

Given that only 100,000 of the millions of common SNPs in the genome have been genotyped, the two SNPs in SYNPR and PDGFC are likely presumed “tag” SNPs, and the functional mutations are other variants located nearby in the genome. To discover the functional mutations, genotyping data for a set of individuals of central and northern European ancestry in Utah from the International HapMap Project (13) was examined. SNP rs10510899 is in linkage disequilibrium (LD, measured by an appreciable pairwise r² correlation) with SNPs spanning approximately 50 kb. This region consists primarily of intronic sequence for SYNPR, along with one coding exon having no known variant. Only two SNPs among the set of 100,000 that we genotyped fall in this region. Neither of these is associated with AMD independently, but they exhibit association in interaction with the 10q26 haplotype. Only the evidence for interaction with rs10510899 exceeds our strict Bonferroni threshold. In the Michigan sample three additional SNPs near rs10510899 were genotyped to see if they also interact with the 10q26 haplotype. Only one, rs6796563, showed a slightly stronger interaction than rs10510899. This SNP is in the same intron as rs10510899, and is in weak linkage disequilibrium with rs10510899 (D′=0.37 r²=0.05).

In contrast, SNP rs997955 is in LD with a larger number of SNPs spanning approximately 225 kb. This region includes intronic sequence of PDGFC, several exonic sequences of PDGFC, and the region downstream of the PDGFC gene, but does not overlap with any other known transcribed sequence. Out of the 100,000 genotyped, twenty-five mapped this region. None are independently associated with AMD, while two show interactions with the 10q26 haplotype. Among these, the only evidence for interaction that exceeded the strict threshold was with rs997955. In the Michigan sample four additional SNPs near rs997955 were genotyped. None of these shows a stronger interaction than rs997955. Therefore, the functional SNPs associated with AMD appear to reside in the SYNPR and PDGFC genes.

To evaluate whether the three interacting genes (LOC387715, SYNPR, and PDGFC) are expressed in the affected target tissue(s), total RNA from human retinal pigment epithelium (RPE), retina, and other tissues for RT-PCR analysis was used. SYNPR transcripts are detected at low levels in native human RPE, but strongly expressed in the retina and placenta PDGFC is expressed at high levels in both native human RPE and cultured RPE as well as in the retina, placenta, and liver. Only low expression of LOC387715 is observed in the retina and cultured RPE. Examination of protein expression in eye tissues using commercially available antibodies against synaptoporin was also performed. The inner plexiform layer showed the strongest labeling for synaptoporin antibodies; nonetheless, the outer plexiform layer was also labeled though the signal is was weaker. This is consistent with the previously-reported localization of synaptoporin in the rabbit retina(14). The distribution of synaptoporin in the horizontal cell presynaptic terminals presumably is involved in synaptic vesicle release. Since these cells provide an inhibitory input that contributes to the antagonistic center-surround responses of the bipolar neurons, alteration of the efficacy of this input could lead to abnormal levels of photoreceptor synaptic activity and consequent cell damage. PDGFC is part of a regulatory cascade that controls activity of matrix metalloproteinases and their tissue inhibitors, molecules intimately involved in regulating vascular quiescence and growth in the eye as well as other tissues (15). The interaction of LOC387715 with two presumably unrelated genes—SYNPR and PDGFC—suggests a common regulatory function in retina or RPE. Preliminary results indicate PDGFC distribution in the inner nuclear layer and ganglion cells of normal retina (data not shown); neither SYNPR nor PDGFC is detectable on the CFH positive drusen.

To provide an overall understanding of the invention, certain illustrative embodiments are described, including. compositions and methods for identifying or aiding in identifying individuals at risk for developing AMD, as well as for diagnosing or aiding in the diagnosis of AMD. However, it will be understood by one of ordinary skill in the art that the compositions and methods described herein may be adapted and modified as is appropriate for the application being addressed and that the compositions and methods described herein may be employed in other suitable applications, and that such other additions and modifications will not depart from the scope hereof.

### REFERENCES

1. Klein R J, Zeiss C, Chew E Y, et al. Complement factor H polymorphism in age-related macular degeneration. Science 2005; 308(5720):385-9.
2. Fisher S A, Abecasis G R, Yashar B M, et al. Meta-analysis of genome scans of age-related macular degeneration. Hum Mol Genet. 2005; 14(15):2257-64.
3. Haines J L, Hauser M A, Schmidt S, et al. Complement factor H variant increases the risk of age-related macular degeneration. Science 2005; 308(5720):419-21.
4. Edwards A O, Ritter R, 3rd, Abel K J, Manning A, Panhuysen C, Farrer La. Complement factor H polymorphism and age-related macular degeneration. Science 2005; 308(5720):421-4.
5. Hageman G S, Anderson D H, Johnson L V, et al. A common haplotype in the complement regulatory gene factor H (HF1/CFH) predisposes individuals to age-related macular degeneration. Proc Natl Acad Sci USA 2005; 102(20):7227-32.
6. Zareparsi S, Branham K E, Li M, et al. Strong Association of the Y402H Variant in Complement Factor H at 1q32 with Susceptibility to Age-Related Macular Degeneration. Am J Hum Genet. 2005; 77(1):149-53.
7. Conley Y P, Thalamuthu A, Jakobsdottir J, et al. Candidate gene analysis suggests a role for fatty acid biosynthesis and regulation of the complement system in the etiology of age-related maculopathy. Hum Mol Genet. 2005.
8. Rivera A, Fisher S A, Fritsche L G, et al. Hypothetical LOC387715 is a second major susceptibility gene for age-related macular degeneration, contributing independently of complement factor H to disease risk. Hum Mol Genet. 2005; 14(21):3227-36.
9. Jakobsdottir J, Conley Y P, Weeks D E, Mah T S, Ferrell R E, Gorin I B. Susceptibility genes for age-related maculopathy on chromosome 10q26. Am J Hum Genet. 2005; 77(3):389407.
10. Hoh J, Wille A, Zee R, et al. Selecting SNPs in two-stage analysis of disease association data: a model-free approach. Ann Hum Genet. 2000; 64:413-7.
11. Cockerham C C. An Extension of the Concept of Partitioning Hereditary Variance for Analysis of Covariates Among Relatives When Epistasis is Present. Genetics 1954; 39:859-82.
12. Majewski J, Schultz D W, Weleber R G, et al. Age-related macular degeneration—a genome scan in extended families. Am J Hum Genet. 2003; 73:540-50.
13. Altshuler D, Brooks L D, Chakravarti A, Collins F S, Daly M J, Donnelly P. A haplotype map of the human genome. Nature 2005; 437(7063):1299-320.
14. Brandstatter J H, Lohrke S, Morgans C W, Wassle H. Distributions of two homologous synaptic vesicle proteins, synaptoporin and synaptophysin, in the mammalian retina. J Comp Neurol 1996; 370(1):1-10.
15. Li X, Ponten A, Aase K, et al. PDGF-C is a new protease-activated ligand for the PDGF alpha-receptor. Nat Cell Biol 2000; 2(5):302-9.
16. Seddon J M, Cote J, Page W F, Aggen S H, Neale M C. The US twin study of age-related macular degeneration: relative roles of genetic and environmental influences. Arch Opthalmol 2005; 123(3):321-7.

TABLE 1
Definitions of the four classes of epistasis.

| | | |
|---|---|---|
| A/A | A/a | a/a |

Type I - Additive by Additive Interaction

| | | | |
|---|---|---|---|
| B/B | 3 | 2 | 1 |
| B/b | 2 | 2 | 2 |
| b/b | 1 | 2 | 3 |

Type II - Additive by Dominant Interaction

| | | | |
|---|---|---|---|
| B/B | 3 | 1 | 3 |
| B/b | 2 | 2 | 2 |
| b/b | 1 | 3 | 1 |

Type III - Dominant by Additive Interaction

| | | | |
|---|---|---|---|
| B/B | 3 | 2 | 1 |
| B/b | 1 | 2 | 3 |
| b/b | 3 | 2 | 1 |

Type IV - Dominant by Dominant Interaction

| | | | |
|---|---|---|---|
| B/B | 1 | 2 | 1 |
| B/b | 2 | 1 | 2 |
| b/b | 1 | 2 | 1 |

TABLE 2
Evidence of association and odds ratios (ORs). Note that the risk factor for
calculating the ORs may be the combination of two classes from the χ² test.
Population A is the AREDS sample; M is the Michigan sample.

| Population | Test | N | χ²P-value | Df | Risk factor | OR (95% CI) |
|---|---|---|---|---|---|---|
| A | rs10490924 | 146 | 0.041 | 2 | At least one risk allele | 2.3 (1.1-4.6) |
| A | Haplotype classes | 146 | 0.0018 | 2 | At least one risk haplotype | 3.7 (1.8-7.9) |
| A | 10q26Hap × rs10510899 (type I) | 146 | 1.3e−08 | 2 | Medium- or high-risk class | 22 (6.2-81) |
| A | 10q26Hap × rs 997955 (type III) | 140 | 4.5e−08 | 2 | Medium- or high-risk class | 9.1 (4.0-22) |
| A | Risk factor sum | 136 | 1.1e−07 | 1 | Sum >= 3 | 7.9 (3.5-18) |
| M | rs10490924 | 367 | 1.1e−08 | 2 | At least one risk allele | 3.2 (2.0-4.8) |
| M | Haplotype classes | 367 | 0.000037 | 2 | At least one risk haplotype | 1.8 (1.2-2.9) |
| M | 10q26Hap × rs10510899 (type I) | 365 | 0.017 | 2 | Medium- or high-risk class | 1.6 (1.0-2.7) |
| M | 10q26Hap × rs997955 (type III) | 357 | 0.00044 | 2 | Medium- or high-risk class | 1.8 (1.1-7.8) |
| M | Risk factor sum | 345 | 1.2e−08 | 1 | Sum >= 3 | 3.7 (2.3-5.8) |

TABLE 3
Haplotypes surrounding rs10490924. The χ² statistic for these 292 observed
haplotypes is 12.5. With 3 degrees of freedom, this translates to a p-value of 0.006.

| Haplotype | rs10490922 | rs10490923 | rs2736911 | rs10490924 | Case | Control |
|---|---|---|---|---|---|---|
| N1 | A | A | C | G | 28 | 13 |
| N2 | T | G | T | G | 32 | 12 |
| N3 | T | G | C | T | 68 | 21 |
| N4 | T | G | C | G | 64 | 54 |

TABLE 4
Observed genotype counts for 10q26Hap, rs10510899, and rs997955.
Counts are given for each of the nine possible genotype pairs.

| 10q26Hap | | | | | | | |
|---|---|---|---|---|---|---|---|
| Cases | | | | Controls | | | |
| AA | AB | BB | Total | AA | AB | BB | Total |

AREDS

| | | | | | | | | | |
|---|---|---|---|---|---|---|---|---|---|
| rs10510899 | AA | 3 | 32 | 14 | 49 | 21 | 11 | 3 | 35 |
| | AB | 15 | 17 | 9 | 41 | 2 | 6 | 4 | 12 |
| | BB | 1 | 5 | 0 | 6 | 1 | 2 | 0 | 3 |
| | Total | 19 | 54 | 23 | 96 | 24 | 19 | 7 | 50 |
| rs997955 | AA | 11 | 45 | 20 | 76 | 23 | 15 | 3 | 41 |
| | AB | 8 | 7 | 1 | 16 | 0 | 2 | 4 | 6 |
| | BB | 0 | 0 | 0 | 0 | 1 | 0 | 0 | 1 |
| | Total | 19 | 52 | 21 | 92 | 24 | 17 | 7 | 48 |

Michigan

| | | | | | | | | | |
|---|---|---|---|---|---|---|---|---|---|
| rs10510899 | AA | 26 | 50 | 36 | 112 | 42 | 52 | 14 | 108 |
| | AB | 18 | 26 | 23 | 67 | 18 | 24 | 10 | 52 |
| | BB | 1 | 3 | 6 | 10 | 4 | 10 | 2 | 16 |
| | Total | 45 | 79 | 65 | 189 | 64 | 86 | 26 | 176 |
| rs997955 | AA | 34 | 65 | 56 | 155 | 55 | 69 | 22 | 146 |
| | AB | 8 | 9 | 7 | 24 | 9 | 15 | 4 | 28 |
| | BB | 0 | 2 | 0 | 2 | 1 | 1 | 0 | 2 |
| | Total | 42 | 76 | 63 | 181 | 65 | 85 | 26 | 176 |

1. An isolated polynucleotide for the detection of a variant gene that is correlated with the occurrence of age related macular degeneration in humans, comprising a nucleic acid molecule selected from the group consisting of:
(a) a nucleic acid molecule that specifically detects a variation in the LOC387715 gene that is correlated with age related macular degeneration in humans;
(b) a nucleic acid molecule that specifically detects a variation in the SYNPR gene that is correlated with the occurrence of age related macular degeneration in humans; and
(c) a nucleic acid molecule that specifically detects a variation in the PDGFC gene that is correlated with the occurrence of age related macular degeneration in humans.

2. The polynucleotide of claim 1, wherein the polynucleotide is a probe that hybridizes, under stringent conditions, to a variation selected from the group consisting of:
(a) a variation in the LOC387715 gene that is correlated with the occurrence of age related macular degeneration in humans;
(b) a variation in the SYNPR gene that is correlated with the occurrence of age related macular degeneration in humans; and
(c) a variation in the PDGFC gene that is correlated with age related macular degeneration in humans.

3. The polynucleotide of claim 2, wherein the variation encodes an amino acid other than alanine at position 69 of the LOC387715 protein.

4. The polynucleotide of claim 3, wherein the variation encodes serine at position 69 of the LOC387715 protein.

5-24. (canceled)

25. The method of claim 31, wherein the variant gene encodes an amino acid other than alanine at position 69 of the LOC387715 protein.

26. The method of claim 31, wherein the variant gene encodes serine at position 69 of the LOC387715 protein.

27-30. (canceled)

31. A method of identifying an individual at risk for developing age related macular degeneration, comprising detecting the presence of a variant gene or polypeptide that is correlated with the occurrence of age related macular degeneration in humans, wherein the variant gene or polypeptide is selected from the group consisting of: a variant LOC387715 gene or polypeptide; a variant SYNPR gene or polypeptide; and a variant PDGFC gene or polypeptide, and wherein the presence of the variant gene or polypeptide indicates that the individual is at risk for developing age related macular degeneration.

32. The method of claim 31, wherein the detecting step comprises:
(a) combining a sample obtained from the individual with (1) a polynucleotide probe that hybridizes, under stringent conditions, to a variation in the LOC387715 gene that is correlated with the occurrence of age related macular degeneration in humans, but does not hybridize to a LOC387715 gene that does not contain the variation; (2) a polynucleotide probe that hybridizes, under stringent conditions, to a variation in the SYNPR gene that is correlated with the occurrence of age related macular degeneration in humans, but does not hybridize to a SYNPR gene that does not contain the variation; and (3) a polynucleotide probe that hybridizes, under stringent conditions, to a variation in the PDGFC gene that is correlated with the occurrence of age related macular degeneration in humans, but does not hybridize to a PDGFC gene that does not contain the variation; and
(b) determining whether hybridization occurs, wherein the occurrence of hybridization of the probes of (a)(1)-(a)(3) indicates that the individual is at risk for developing age related macular degeneration.

33. A diagnostic kit for detecting a variant gene correlated with age related macular degeneration in a sample from an individual, comprising: the polynucleotide of claim 1; a container; and
a label and/or instructions for the use of the diagnostic kit in the detection of variant genes in a sample.

34. A composition for treating a subject suffering from or at risk for age related macular degeneration, comprising: (a) an effective amount of (1) an isolated or recombinantly produced wildtype LOC387715 polypeptide, or a fragment thereof; (2) an isolated or recombinantly produced wildtype SYNPR polypeptide, or a fragment thereof; or (3) an isolated or recombinantly produced wildtype PDGFC polypeptide, or a fragment thereof; and (b) a pharmaceutically acceptable carrier.

35. A method of treating a subject suffering from or at risk for age related macular degeneration, comprising administering to the subject an effective amount of the composition of claim 34.

36. A composition for treating a subject suffering from or at risk for age related macular degeneration, comprising: (a) an effective amount of (1) an isolated or recombinantly produced nucleic acid molecule coding for a LOC387715 polypeptide, or a fragment thereof; (2) an isolated or recombinantly produced nucleic acid molecule coding for a SYNPR polypeptide, or a fragment thereof; or (3) an isolated or recombinantly produced nucleic acid molecule coding for a PDGFC polypeptide, or a fragment thereof; and (b) a pharmaceutically acceptable carrier.

37. A method of treating a subject suffering from or at risk for age related macular degeneration, comprising administering to the subject an effective amount of the composition of claim 36.

38. (canceled)

39. A composition for treating a subject suffering from or at risk for age related macular degeneration, comprising:
(a) a nucleic acid molecule comprising an antisense sequence that hybridizes to (i) a variant LOC387715 gene or mRNA that is correlated with the occurrence of age related macular degeneration in humans,
(ii) a variant SYNPR gene or mRNA that is correlated with the occurrence of age related macular degeneration in humans, or
(iii) a variant PDGFC gene or mRNA that is correlated with the occurrence of age related macular degeneration in humans; and
(b a pharmaceutically acceptable carrier.

40-41. (canceled)

42. A method for treating a subject suffering from or at risk for age related macular degeneration, comprising administering to the subject an effective amount of the composition of claim 39.

43. A composition for treating a subject suffering from or at risk for age related macular degeneration, comprising:
(a) a nucleic acid molecule comprising a siRNA or miRNA sequence, or a precursor thereof, that hybridizes to (i a variant LOC387715 gene or mRNA that is correlated with the occurrence of age related macular degeneration in humans;
(ii) a variant SYNPR gene or mRNA that is correlated with the occurrence of age related macular degeneration in humans; or
(iii) a variant PDGFC gene or mRNA that is correlated with the occurrence of age related macular degeneration in humans; and
(b) a pharmaceutically acceptable carrier.

44-45. (canceled)

46. A method for treating a subject suffering from or at risk for age related macular degeneration, comprising administering to the subject an effective amount of the composition of claim 43.

47. The method of claim 31, further comprising detecting the presence of a variant CFH gene that is correlated with the occurrence of age related macular degeneration.

48. (canceled)

49. The diagnostic kit of claim 33, further comprising a polynucleotide probe that hybridizes, under stringent conditions, to a variation in a CFH gene that is correlated with the occurrence of age related macular degeneration.

50. The composition of claim 34, further comprising an effective amount of an isolated or recombinantly produced wildtype CFH polypeptide, or a fragment thereof.

51. The method of claim 35, further comprising, prior to the administering step, the step of detecting the presence or absence of a risk variation at an LOC387715 polymorphic site in a sample from the patient, wherein the presence of a risk variation at the LOC387715 polymorphic site indicates the patient has or is at risk of developing AMD.
